(19) United States
(12) Patent Application Publication
(10) Pub. No.: US 2017/0332988 A1
(43) Pub. Date: Nov. 23, 2017

(54) BREAST IMAGING APPARATUS

(71) Applicant: CANON KABUSHIKI KAISHA, Tokyo (JP)

(72) Inventors: Osamu Tsujii, Kawasaki-shi (JP); Tetsuo Shimada, Hachioji-shi (JP); Sakiko Yamaguchi, Tokyo (JP); Takahiro Noguchi, Tokyo (JP)

(21) Appl. No.: 15/533,107

(22) PCT Filed: Jun. 22, 2016
(86) PCT No.: PCT/JP2016/003014

§ 371 Date: Jun. 5, 2017

(30) Foreign Application Priority Data
Jun. 30, 2015 (JP) 2015-131836

(51) Int. Cl. A61B 6/00; A61B 6/04; A61B 6/03

(52) U.S. Cl. CPC A61B 6/502; A61B 6/588; A61B 6/4405; A61B 6/032; A61B 6/0414; A61B 6/4085

(57) ABSTRACT

A breast imaging apparatus includes a radiation generation unit configured to generate radiation and a radiation detection unit configured to detect radiation irradiation from the radiation generation unit and can rotate the radiation generation unit and the radiation detection unit in a state in which they face each other. Imaging is performed in a state in which a body part (breast) of an object to be imaged is sandwiched by a pressing panel on a first side of the breast imaging apparatus. In addition, imaging is performed while rotating the radiation generation unit and the radiation detection unit in a state in which the body part (breast) of the object to be imaged is inserted between the radiation generation unit and the radiation detection unit from a second side opposite to the first side of the breast imaging apparatus.

### TECHNICAL FIELD

The present invention relates to a breast imaging apparatus that performs mammography using radiation.

### BACKGROUND ART

As a breast imaging apparatus, there exists an apparatus that captures a breast using a radiation generation unit configured to generate radiation and a radiation detection unit configured to detect the radiation. In addition, both mammogram imaging and CT imaging are performed by a single breast imaging apparatus (for example, Japanese Patent Laid-Open No. 2013-538668).

In the breast imaging apparatus of JP2013-538668, when performing CT imaging, a breast of a patient is fixed between two plates, as in mammogram imaging. It is therefore difficult to do CT imaging in a state in which the breast is appropriately held.

### SUMMARY OF INVENTION

One embodiment of the present invention provides a breast imaging apparatus capable of performing both mammogram imaging and CT imaging in a state in which a breast of a patient is appropriately held.

According to one aspect of the present invention, there is provided a breast imaging apparatus including a radiation generation unit configured to generate radiation and a radiation detection unit configured to detect radiation irradiation from the radiation generation unit and capable of rotating the radiation generation unit and the radiation detection unit in a state in which the radiation generation unit and the radiation detection unit face each other, comprising: first imaging means for performing mammogram imaging using a pressing panel on a first side of the breast imaging apparatus; and second imaging means for performing CT imaging while rotating the radiation generation unit and the radiation detection unit on a second side opposite to the first side of the breast imaging apparatus.

According to another aspect of the present invention, there is provided a breast imaging apparatus including a radiation generation unit configured to generate radiation and a radiation detection unit configured to detect radiation irradiation from the radiation generation unit and capable of rotating the radiation generation unit and the radiation detection unit in a state in which the radiation generation unit and the radiation detection unit face each other, comprising: first imaging means for performing mammogram imaging while pressing a breast on a first side of the breast imaging apparatus; and second imaging means for performing CT imaging on a second side opposite to the first side of the breast imaging apparatus.

According to another aspect of the present invention, there is provided a breast imaging apparatus including a radiation generation unit configured to generate radiation and a radiation detection unit configured to detect radiation irradiation from the radiation generation unit and capable of rotating the radiation generation unit and the radiation detection unit in a state in which the radiation generation unit and the radiation detection unit face each other, comprising: first imaging means for performing imaging in a state in which a breast of an object is pressed by a pressing panel on a first side of the breast imaging apparatus; and second imaging means for performing imaging while rotating the radiation generation unit and the radiation detection unit in a state in which the breast of object is inserted between the radiation generation unit and the radiation detection unit on a second side opposite to the first side of the breast imaging apparatus.

Further features of the present invention will become apparent from the following description of exemplary embodiments with reference to the attached drawings.

According to the present invention, it is possible to perform both mammogram imaging and CT imaging in a state in which a breast of a patient is appropriately held.

### BRIEF DESCRIPTION OF DRAWINGS

FIG. 1 is a view showing the outer appearance of a breast imaging apparatus according to an embodiment at the time of mammogram imaging;

FIG. 2 is a view showing the outer appearance of the breast imaging apparatus according to the embodiment at the time of mammogram imaging;

FIG. 3 is a view showing the outer appearance of the breast imaging apparatus according to the embodiment at the time of CBCT imaging;

FIG. 4 is a view showing the outer appearance of the breast imaging apparatus according to the embodiment at the time of CBCT imaging;

FIG. 5A is a view for explaining the imaging operation of the breast imaging apparatus according to the first embodiment;

FIG. 5B is a view for explaining the imaging operation of the breast imaging apparatus according to the first embodiment;

FIG. 6A is a view for explaining the imaging operation of a breast imaging apparatus according to the second embodiment;

FIG. 6B is a view for explaining the imaging operation of a breast imaging apparatus according to the second embodiment;

FIG. 6C is a view for explaining the imaging operation of a breast imaging apparatus according to the second embodiment;

FIG. 7 is a view showing the outer appearance of a breast imaging apparatus according to the third embodiment;

FIG. 8 is a view showing the outer appearance of the breast imaging apparatus according to the present invention viewed from the CT imaging side;

FIG. 9 is a sectional view of the breast imaging apparatus according to the third embodiment;

FIG. 10 is a view showing the outer appearance of the breast imaging apparatus according to the present invention viewed from the mammogram imaging side;

FIG. 11 is a block diagram showing the arrangement of the breast imaging apparatus according to the third embodiment;

FIG. 12 is a view showing the arrangement of the vertical driving unit of the breast imaging apparatus according to the third embodiment;

FIG. 13 is a view showing a rotation form by the rotation driving unit of the breast imaging apparatus according to the third embodiment; and

FIG. 14A is a view showing a state in which the front cover is removed from the gantry of the breast imaging apparatus according to the third embodiment.

FIG. 14B is a view showing a state in which the front cover is removed from the gantry of the breast imaging apparatus according to the third embodiment.

### DESCRIPTION OF EMBODIMENTS

Several preferred embodiments of the present invention will now be described in detail with reference to the accompanying drawings.

### First Embodiment

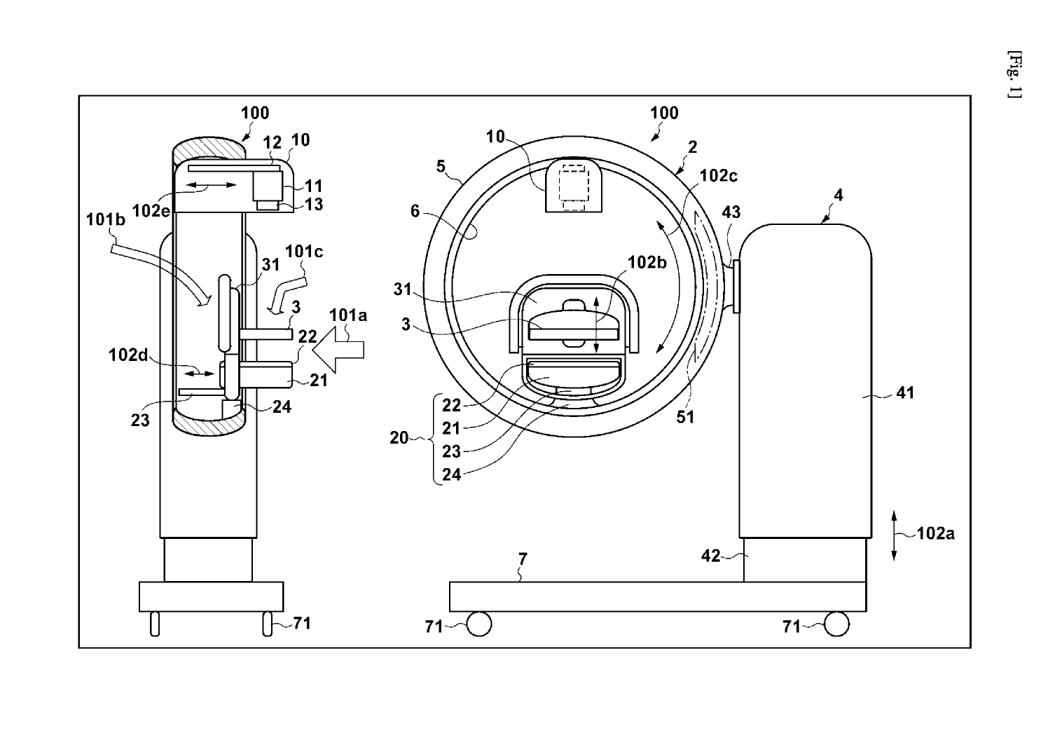

FIG. 1 is a view showing the outer appearance of a breast imaging apparatus 100 according to the first embodiment. In the first embodiment, a description will be made using the breast imaging apparatus 100 capable of performing mammogram imaging and CT imaging as a radiation imaging apparatus. The breast imaging apparatus 100 includes a radiation imaging unit 2 including

a radiation generation unit 10 that includes a radiation tube 11 (for example, an X-ray tube) serving as a radiation source and generates radiation,

a radiation detection unit 20 that includes a radiation detector 21 such as an FPD (Flat Panel Detector) and detects radiation irradiation from the radiation generation unit 10, and

a rotation unit (also called a gantry) that can rotate the radiation generation unit 10 and the radiation detection unit 20 in a state in which they face each other. The rotation unit of the radiation imaging unit 2 includes, for example, a ring-shaped rotation frame 6 in which the radiation generation unit 10 and the radiation detection unit 20, which face each other, are arranged and fixed, and a fixed frame 5 that rotatably stores the rotation frame 6.

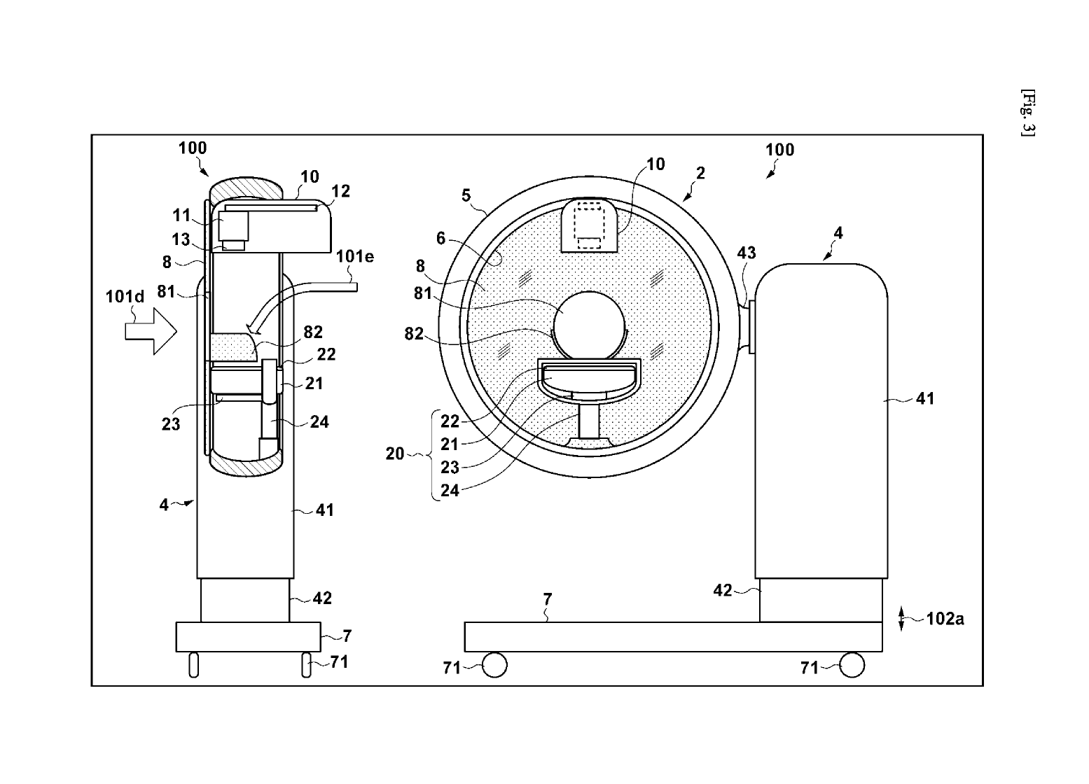

For mammogram imaging, the radiation imaging unit 2 is configured to insert a breast of an object as a body part to be imaged between a pressing panel 3 and the radiation detection unit 20 from the first side (the side of an arrow 101a) of the surface of revolution of the rotation frame 6. For CT imaging (in this embodiment, CBCT imaging), the radiation imaging unit 2 is configured to insert the body part to be imaged between the radiation generation unit 10 and the radiation detection unit 20 from the second side (an arrow 101d in FIG. 3) opposite to the first side of the surface of revolution of the rotation frame 6. That is, the radiation imaging unit 2 of the breast imaging apparatus 100 can implement

a first imaging mode to perform imaging in a state in which the breast of the object is pressed by the pressing panel 3 on the first side of the breast imaging apparatus 100, and

a second imaging mode to perform imaging while rotating the radiation generation unit 10 and the radiation detection unit 20 in a state in which the breast of the object is inserted between the radiation generation unit 10 and the radiation detection unit 20 on the second side opposite to the first side of the breast imaging apparatus 100. Imaging of the breast of the object inserted from the first side is mammogram imaging, and imaging of the breast of the object inserted from the second side is CT imaging of the breast. The gantry rotated in a state in which the radiation generation unit 10 and the radiation detection unit 20 face each other has a space that allows the operator to access, from the first side, the breast inserted from the second side and captured in the second imaging mode.

FIG. 1 shows a state in which the breast imaging apparatus 100 captures a CC (Caranio Caudal) view of a mammogram. The rotation position of the rotation frame 6 is decided such that the radiation tube 11, the pressing panel 3, and the radiation detector 21 are arranged in the vertical direction. A pressing panel support unit 31 supports the pressing panel 3, and moves the pressing panel 3 in a predetermined direction 102b (for example, a direction in which the pressing panel support unit 31 mounted on the rotation frame 6 moves toward the rotation center of the rotation frame 6). The pressing panel support unit 31 is placed to be removable from the rotation frame 6. Note that the pressing panel support unit 31 may be placed to be removable from a constituent element integrated with the rotation frame 6, for example, the radiation detection unit 20, a detector moving unit 23, or an elevating unit 24. An imaging technician can remove the pressing panel support unit 31 together with the pressing panel 3. The imaging technician can adjust the distance between the pressing panel 3 and the radiation detection unit 20 by moving the pressing panel 3 by the pressing panel support unit 31. The breast of the object can be pressed by moving the pressing panel 3. In mammogram imaging, the breast arranged between the pressing panel 3 and the radiation detection unit 20 is pressed between the pressing panel 3 and the radiation detection unit 20 and undergoes radiation imaging.

The fixed frame 5 of the radiation imaging unit 2 is supported by a movable column portion 41 of a column 4 via a fixed shaft 43. In the column 4, the movable column portion 41 can move vertically with respect to a fixed column portion 42. The radiation imaging unit 2 including the radiation generation unit 10 and the radiation detection unit 20 is thus supported by the column 4 to be movable in the vertical direction (arrow 102a). The column 4 (fixed column portion 42) is fixed to a cart 7 with casters 71, thereby providing portability to the breast imaging apparatus 100. Note that the column 4 need not always be fixed to the cart 7 and may be fixed on the floor.

An arcuate rotation motor 51 (linear motor) is attached to the distal end of the fixed shaft 43 that connects the column 4 and the radiation imaging unit 2. The rotation frame 6 is rotatably connected to the rotation motor 51 via a bearing. The fixed frame 5 is stationarily connected to the fixed shaft 43 and includes the rotation frame 6. The bearing is arranged in the gap between the fixed frame 5 and the rotation frame 6. When the rotation motor 51 is driven, the rotation frame 6 rotates with respect to the fixed frame 5, as indicated by an arrow 102c.

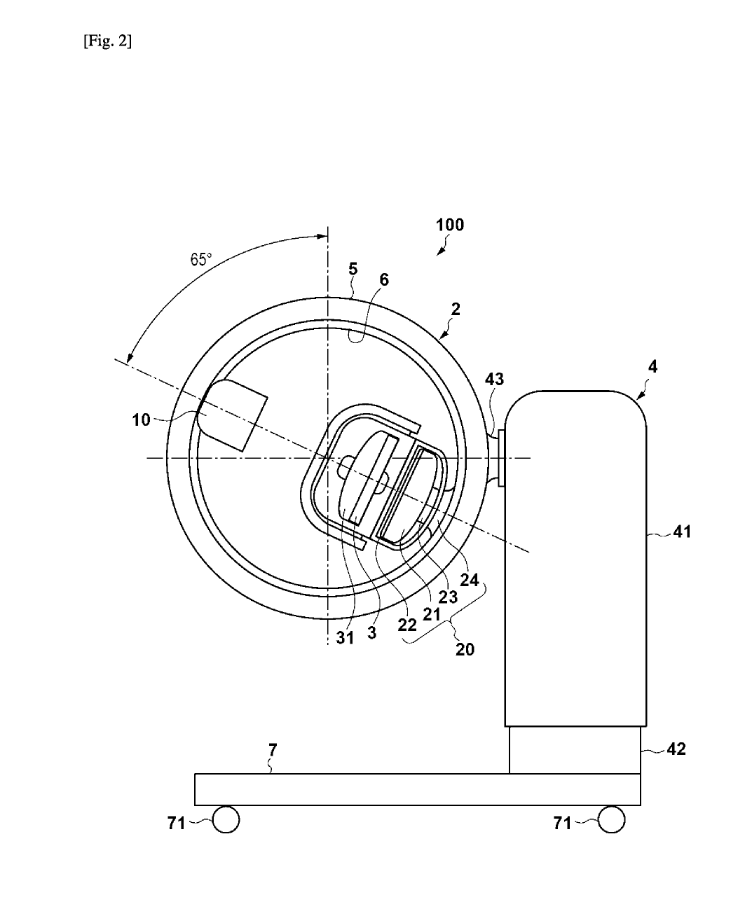

When capturing a CC view of a mammogram, the radiation tube 11, the radiation detector 21, and the pressing panel 3 are arranged in the vertical direction, as shown in FIG. 1. On the other hand, when capturing an MLO (Mediolateral Oblique) view of a mammogram by the breast imaging apparatus 100, the rotation frame 6 is rotated by a predetermined angle (for example, about 65°) from the state shown in FIG. 1 and stopped, as shown in FIG. 2. Note that the stop state of the rotation frame 6 may be maintained by servo control or a brake. The breast that is the body part to be imaged is pressed between the radiation detector 21 and the pressing panel 3 and undergoes radiation imaging. By capturing such an MLO view, imaging of an armpit can be performed.

Referring back to FIG. 1, in mammogram imaging, the imaging technician accesses the breast of the object via the hollow portion of the rotation frame 6, as indicated by an arrow 101b, arranges the breast between the pressing panel 3 and the radiation detector 21 of the breast imaging apparatus 100, and adjusts the pressing. On the first side that is the breast insertion side upon mammogram imaging, the radiation tube 11, the radiation detector 21, and the pressing panel 3 are fixed such that they project in a first direction with respect to the surface of revolution of the rotation frame 6. For this reason, the imaging technician can also access the breast of the object from a side (between the surface of revolution and the object) of the breast imaging apparatus 100, as indicated by an arrow 101c, and adjust the pressing.

The arrangement of the breast imaging apparatus 100 at the time of mammogram imaging has been described above. CT imaging by the breast imaging apparatus 100 will be described next. The radiation generation unit 10 includes a radiation source moving unit 12 that can move and arrange the radiation tube 11 in the rotation axis direction (arrow 102e) of the rotation frame 6 for mammogram imaging and CT imaging. The radiation source moving unit 12 includes, for example, a rail on which the radiation tube 11 slides, and the imaging technician can manually move the radiation tube 11. Alternatively, the radiation tube 11 may be moved in the direction of the arrow 102e by the driving force of a linear motor or the like. The radiation detection unit 20 includes the detector moving unit 23 that can move and arrange the radiation detector 21 in the rotation axis direction (arrow 102d) of the rotation frame 6 for mammogram imaging and CT imaging. The detector moving unit 23 includes a rail on which the radiation detector 21 slides, and the imaging technician can move the radiation detector 21 in the direction of the arrow 102d. Alternatively, the radiation detector 21 may be moved in the direction of the arrow 102d by the driving force of a linear motor or the like. The radiation detection unit 20 also includes the elevating unit 24 that moves the radiation detector 21 in the rotation center direction (arrow 102b) of the rotation frame 6 for mammogram imaging and CT imaging.

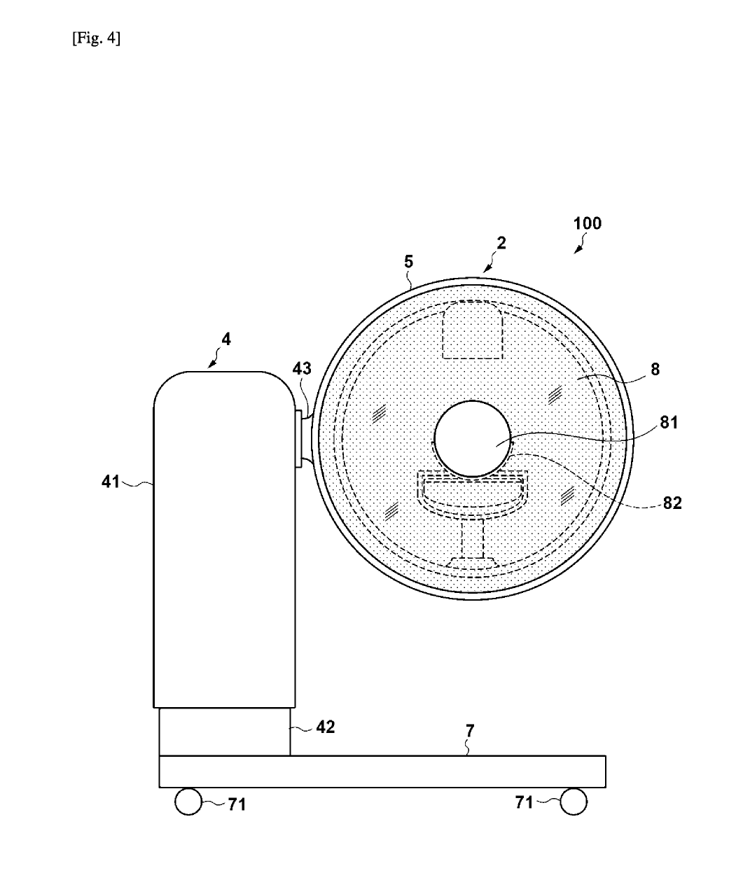

FIG. 3 shows a state in which the breast imaging apparatus 100 according to this embodiment performs CT imaging (in this embodiment, CBCT imaging) of a breast of an object. FIG. 4 is a view showing the outer appearance of the breast imaging apparatus 100 from the direction of the arrow 101d that is the insertion direction of the breast of the object. At the time of CBCT imaging, the breast is inserted from the second side opposite to the breast insertion side (first side) at the time of mammogram imaging (arrow 101d). In addition, the radiation tube 11 and the radiation detector 21 are moved to the second side opposite to the first side and arranged by the radiation source moving unit 12 and the detector moving unit 23. As described above, the radiation source moving unit 12 and the detector moving unit 23 may be configured to move the radiation tube 11 and the radiation detector 21 by motor driving or the like or manually. The radiation source moving unit 12 and the detector moving unit 23 need only be configured to arrange the radiation tube 11 and the radiation detector 21 at positions where mammogram imaging can be executed for a breast inserted from the first side, and CBCT imaging can be executed for a breast inserted from the second side.

Note that the pressing panel support unit 31 and the pressing panel 3 are removable from the rotation frame 6, that is, the radiation imaging unit 2. If the pressing panel support unit 31 and the pressing panel 3 are kept placed on the rotation frame 6, they hinder the imaging technician from accessing the breast of the object when performing CBCT imaging. Hence, at the time of CBCT imaging, the pressing panel support unit 31 is removed from the rotation frame 6 together with the pressing panel 3, as shown in FIGS. 3 and 4.

The elevating unit 24 of the radiation detection unit 20 moves the radiation detector 21 toward the rotation center of the rotation frame 6, thereby changing the distance between the radiation detector 21 and the radiation generation unit 10 (radiation tube 11). The radiation tube 11 and the radiation detector 21 are thus arranged in a positional relationship appropriate for CBCT imaging. A front cover 8 is removably provided on the second side of the radiation imaging unit 2 (fixed frame 5). The front cover 8 is circular, and is placed to be removable from the circular fixed frame 5. Note that the front cover 8 need only be fixed to a member immovable with respect to the rotation of the rotation frame 6, and may be placed on, for example, the fixed shaft 43. The front cover 8 is provided with an opening 81 to insert the breast of the object that is the body part to be imaged. More specifically, the circular opening 81 used to insert the breast of the object is provided at the center of the front cover 8. The front cover 8 includes, around the opening 81, a breast table 82 serving as a support used to support the breast inserted from the opening 81. The lower portion of the breast table 82 is curved, and the upper portion of the breast table 82 is open. Note that in this embodiment, the breast table 82 is fixed to the front cover 8. However, the present invention is not limited to this. For example, the breast table 82 may be fixed to the fixed frame 5 via a support member. During CBCT imaging, radiation images are captured while rotating the rotation frame 6 with respect to the fixed frame 5, and a re-construction unit (not shown) calculates a 3D reconstructed image. The front cover 8 fixed to the fixed frame 5 separates the space of the object (not shown) from the radiation generation unit 10 and the radiation detection unit 20 which rotate during imaging. The breast of the object is held on the breast table 82 and therefore fixed during imaging. As described above, the support form for supporting the breast to be captured in CBCT imaging (second imaging mode) is the breast table 82, which is different from the support form (a support form using the pressing panel 3) for supporting the breast to be captured in mammogram imaging (first imaging form).

Note that in the above-described example, the breast table 82 is connected along the periphery of the opening of the front cover 8. However, the present invention is not limited to this. For example, the breast table 82 need only be held immovably with respect to the rotation of the rotation frame 6 during CBCT imaging, and may be connected to, for example, the fixed frame 5. However, when the breast table 82 is connected to the front cover 8, a support member used to connect the fixed frame 5 and the breast table 82 is unnecessary. Hence, the imaging technician can easily access the breast of the object from the direction of an arrow 101e. To freely three-dimensionally distribute the mammary structure, it is preferable that the breast table 82 does not press the breast. However, the breast may be pressed so much that the three-dimensional distribution is not lost (not shown). In FIG. 3, the front cover 8 has a removable form. However, the front cover 8 may have an opening/closing structure without hindrance to mammogram imaging.

The imaging technician accesses the breast of the object from the first side of the fixed frame 5 and the rotation frame 6 via the opening 81 of the front cover 8, as indicated by the arrow 101e, and places the breast of the object on the breast table 82. Note that the front cover 8 may be transparent on both sides. However, the front cover 8 may be opaque from the object side (the side of the arrow 101d) and transparent from the imaging technician side (the side of the arrow 101a in FIG. 1). The front cover 8 formed to be opaque from the object side can prevent the object from becoming frightened by viewing the movement of the radiation generation unit 10 or the radiation detection unit 20 through the front cover 8. In addition, the front cover 8 formed to be transparent from the imaging technician side facilitates confirmation of the state of the object and access to the breast of the object.

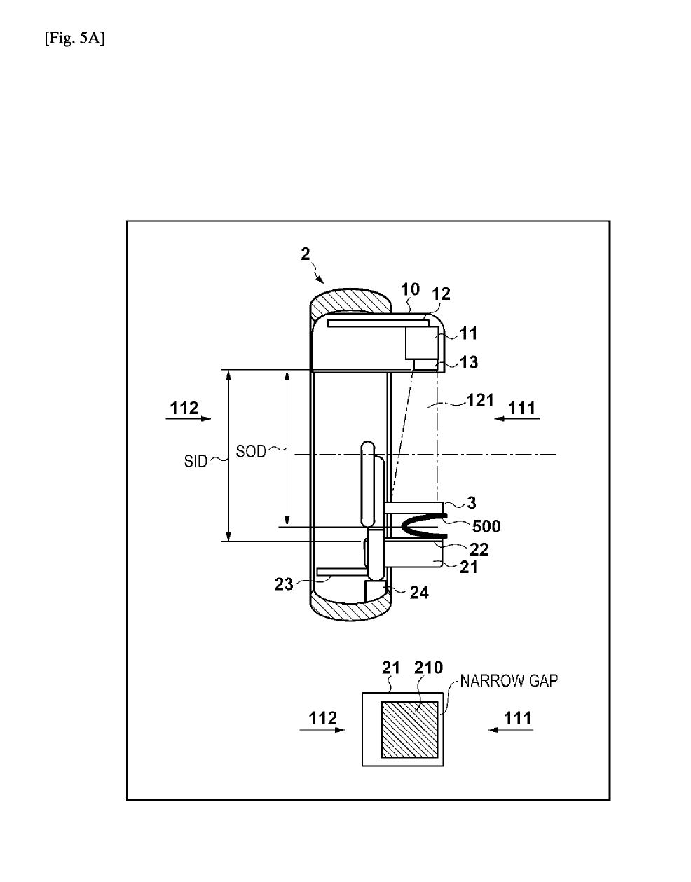

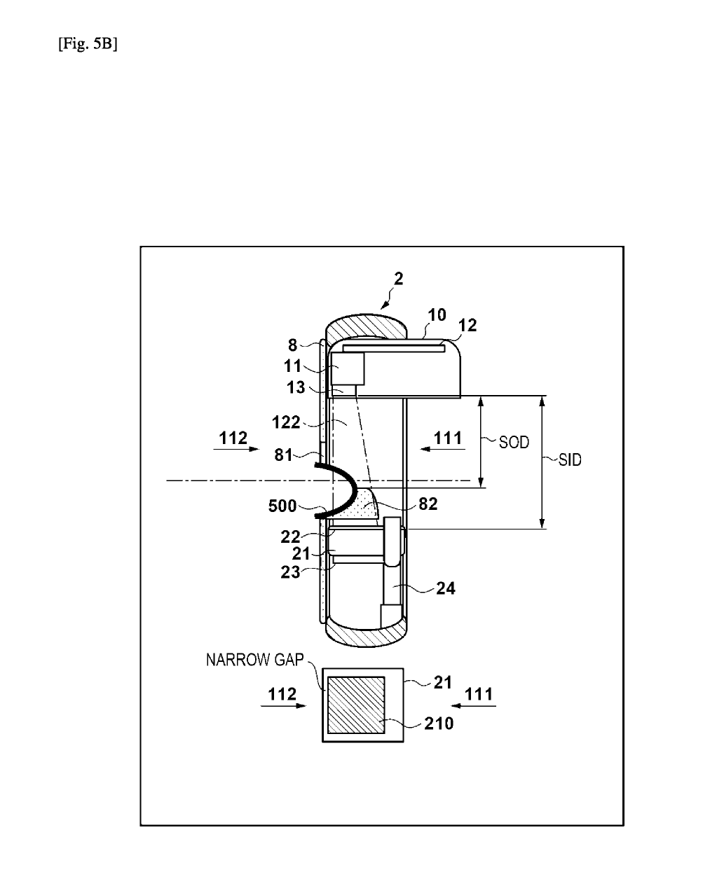

FIGS. 5A and 5B are views showing the mammogram imaging state and the CBCT imaging state of the breast imaging apparatus 100 according to this embodiment. As shown in FIGS. 5A and 5B, the access surface with respect to the rotation frame 6 for the object is reversed between mammogram imaging and CBCT imaging. The side of the access surface for the object at the time of mammogram imaging is defined as a first side 111, and the side of the access surface for the object in CBCT imaging is defined as a second side 112.

FIG. 5A is a side view in mammogram imaging. The object stands on the first side 111. In FIG. 5A, the rotation frame 6 is located at a position corresponding to CC imaging. In MLO imaging, the rotation frame 6 is rotated by about 65° (see FIG. 2). In the radiation generation unit 10, the radiation tube 11 is connected to the rotation frame 6 via the radiation source moving unit 12. The radiation detector 21, the pressing panel support unit 31, the pressing panel 3, and the like are connected to the rotation frame 6 via the elevating unit 24.

By the radiation generation unit 10 and the radiation detection unit 20 including these components, the radiation imaging unit 2 provides different imaging geometric systems in mammogram imaging and CT imaging. Different imaging geometric systems (SID (Source to Image Distance) and SOD (Source to Object Distance)) can thus be provided in mammogram imaging and CBCT imaging. In addition, since the radiation tube 11 and the radiation detector 21 project to the first side 111 with respect to the surface of revolution of the rotation frame 6 and the fixed frame 5, the imaging technician can access a breast 500 of the object from a side in mammogram imaging (the arrow 101c in FIG. 1). In addition, when the front cover 8 placed on the second side 112 is removed, the imaging technician can access the breast 500 of the object from the second side 112 via the hollow portion of the rotation frame 6 (the arrow 101b in FIG. 1).

Additionally, a radiation aperture 13 is placed in front of the radiation tube 11, and a grid 22 for scattered ray reduction is arranged in front of the radiation detector 21. Since the imaging geometric system changes between mammogram imaging and CBCT imaging, the radiation aperture 13 changes the aperture shape in accordance with mammogram imaging or CBCT imaging. Note that deformation of the opening shape of the radiation aperture 13 can be implemented by an arrangement that deforms the opening shape in accordance with a switching operation of the imaging technician or by exchanging the radiation aperture 13. In addition, the stripe direction, stripe frequency, and grid ratio of the grid 22 are also set in accordance with mammogram imaging or CBCT imaging. For example, the imaging technician exchanges the grid between mammogram imaging and CBCT imaging, thereby coping with each imaging mode.

FIG. 5B is a side view in CBCT imaging. In the breast imaging apparatus 100 according to this embodiment, the imaging technician can easily change the form of mammogram imaging shown in FIG. 5A to the form of CBCT imaging shown in FIG. 5B. That is, the imaging technician removes the pressing panel 3 from the breast imaging apparatus 100 shown in FIG. 5A, moves the radiation tube 11 to the second side 112, moves the radiation detector 21 to the second side 112, and moves the radiation detector 21 upward in FIG. 5A by the elevating unit 24. Note that an arrangement for moving the radiation detector 21 in the horizontal direction may be implemented by, for example, rotating the radiation detector about the elevating unit 24. However, if the radiation detector 21 that has rotated interferes with the rotation frame 6, the radiation detector 21 is configured to be rotatable, for example, after it is raised by the elevating unit 24 to the vicinity of the center of the rotation frame 6. To change the form of CBCT imaging shown in FIG. 5B to the form of mammogram imaging shown in FIG. 5A, for example, the imaging technician moves the radiation tube 11 and the radiation detector 21 to the first side 111, moves the radiation detector 21 downward in FIG. 5B by the elevating unit 24, and mounts the pressing panel 3 (pressing panel support unit 31) on the radiation detection unit 20.

In CBCT imaging, the object stands on the second side 112. The breast 500 of the object can be aligned with the opening 81 by vertically moving the movable column portion 41. For example, the radiation imaging unit 2 is moved downward by a distance indicated by an arrow 131, thereby aligning the breast 500 of the object with the opening 81. As described above, the pressing panel support unit 31 and the pressing panel 3 have removable structures and are removed in CBCT imaging. In addition, since the access surface for the object changes between mammogram imaging and CBCT imaging, the radiation tube 11 is placed so as to rotate by 180° when moving from the first side 111 to the second side 112. A radiation beam is formed to reduce the blind area (area that is not imaged) of the chest wall portion of the object small, as indicated by radiation beam shapes 121 and 122 in FIG. 5A and 5B. Since the radiation beam is asymmetrical, the radiation tube 11 needs to be rotated. Note that a radiation beam suitable for each imaging may be formed by the radiation aperture 13 without rotating the radiation tube 11. That is, the radiation generation unit 10 can rotate the radiation shape (the radial shape of radiation) from the radiation tube 11 serving as a radiation source by 180° about the radial direction from the rotation center of the rotation frame 6 between mammogram imaging and CBCT imaging.

The detector moving unit 23 can mount the radiation detector 21 in a state in which the radiation detector 21 is rotated by 180° about the radial direction from the rotation center of the rotation frame 6 between mammogram imaging and CBCT imaging. This is because the access surface for the object changes between mammogram imaging and CBCT imaging. For example, the radiation detector 21 for mammography has a narrow gap (the distance from the outer edge of the sensor to a detection area 210 is 5 mm or less) along only one side of the detection area 210 to reduce the blind area of the chest wall portion, as shown in FIGS. 5A and 5B. For this reason, the radiation detector 21 can be moved and placed so as to rotate by 180° such that the narrow gap side is directed to the object, as shown in FIGS. 5A and 5B. Note that in the arrangement that rotates the radiation detector 21 about the elevating unit 24 to move the radiation detector 21, the narrow gap side is directed to the object by the rotation.

Note that in CBCT imaging, the imaging technician exercises the technique so as to access the breast of the object from the opposite side (first side 111) of the front cover 8 via the opening 81 of the front cover 8 and place most the breast 500 of the object on the breast table 82.

As described above, since the SOD and SID change between mammogram imaging and CBCT imaging, the elevating unit 24 moves the radiation detector 21 in a direction perpendicular to the rotation axis of the rotation frame 6. The SOD is the distance between the radiation focus and the breast center, and the SID is the distance between the radiation focus and the surface of the radiation detector. As examples of the SOD and SID, SOD=60 cm and SID=65 cm in mammogram imaging, and SOD=35 cm and SID=50 cm in CBCT imaging. Note that in addition to or in place of moving the radiation detector 21, the radiation tube 11 may be moved in a direction perpendicular to the rotation axis of the rotation frame 6. However, since the radiation tube 11 is heavier than the radiation detector 21, only the radiation detector 21 is preferably moved to cope with the changing of the SOD and SID without moving the radiation tube 11. In this case, the radiation tube 11 is fixed based on CBCT imaging, and only the radiation detector 21 is moved to cope with the changing of the SOD and SID. A counterbalance (not shown) is placed in the rotation frame 6. The counterbalance is arranged based on the positions of the radiation tube 11 and the radiation detector 21 at the time of CBCT imaging. This is because the rotation speed is required to be stable in CBCT imaging but not required to be stable in mammogram imaging.

The opening shape of the radiation aperture 13 is set to a shape according to CBCT imaging. The stripe direction, stripe frequency, and grid ratio of the grid 22 are set in accordance with CBCT imaging. Note that in CBCT imaging, the grid 22 is placed such that the grid lines extend in a direction perpendicular to a rotation axis 25 of the rotation frame 6. This aims at preventing a ring artifact from being generated in a re-constructed image. In the CBCT imaging, since data may be collected by setting a pixel pitch larger than in mammogram imaging, the grid frequency can be lower than in mammogram imaging. Additionally, in the CBCT imaging, since the imaging tube voltage is higher than in mammogram imaging, the grid ratio of the grid is made larger than in mammogram imaging.

As described above, according to the breast imaging apparatus 100 of the first embodiment, it is possible to easily execute mammogram imaging and CT imaging. For example, if a morbid portion is found by CBCT imaging, mammogram imaging including an armpit to confirm metastasis of a lymph node can be performed by the same apparatus as that for the CBCT imaging. In addition, if a dense breast is found or some kind of disease is suspected by mammogram imaging, CBCT imaging can be performed by the same apparatus as that for the mammogram imaging. It is therefore possible to easily cooperatively perform mammogram imaging and CBCT imaging and raise the throughput of imaging.

### Second Embodiment

In the first embodiment, one radiation tube 11 and one radiation detector 21 are moved for mammogram imaging and CBCT imaging. In the second embodiment, a radiation tube 11 and a radiation detector 21 are arranged for each of mammogram imaging and CBCT imaging to obviate the movement (a radiation source moving unit 12 and a detector moving unit 23) of the radiation tube 11 and the radiation detector 21 in the rotation axis direction of a rotation frame 6.

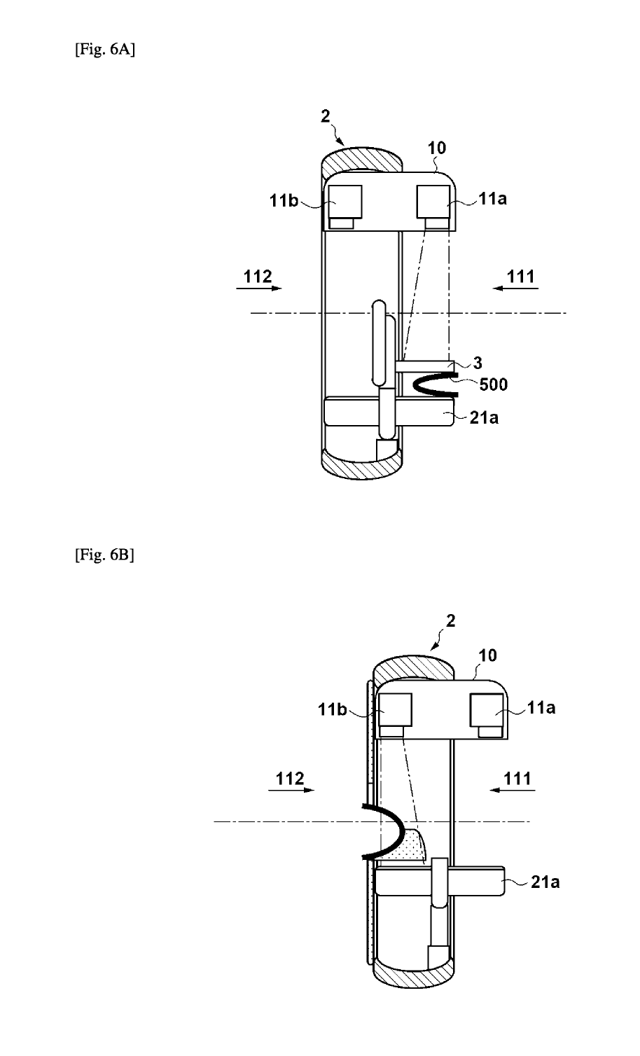

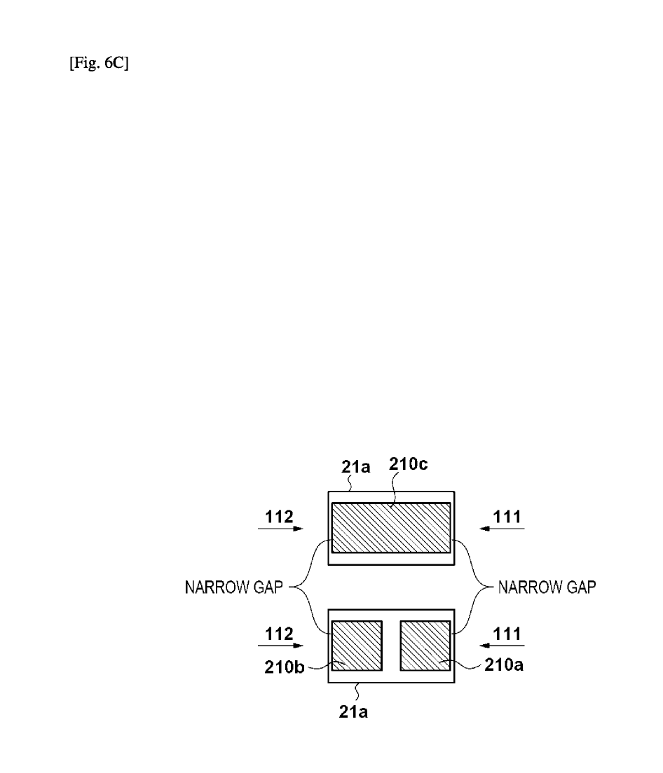

FIG. 6A is a view showing the state of a radiation imaging unit 2 at the time of mammogram imaging of a breast imaging apparatus 100 according to the second embodiment. FIG. 6B is a view showing the state of the radiation imaging unit 2 at the time of CBCT imaging of the breast imaging apparatus 100 according to the second embodiment. A radiation generation unit 10 includes a radiation tube 11a for mammogram imaging and a radiation tube 11b for CBCT imaging. A radiation detector 21a having a large detection area corresponds to both mammogram imaging from a first side 111 and CBCT imaging from a second side 112.

In the breast imaging apparatus 100 according to the second embodiment as described above, to change the mode from the mammogram imaging mode to the CBCT imaging mode, the imaging technician removes a pressing panel 3 (pressing panel support unit 31) and moves a radiation detection unit 20 upward. The imaging technician then switches the radiation tube to be used from the radiation tube 11a to the radiation tube 11b. When changing the mode from the CBCT imaging mode to the mammogram imaging mode, the imaging technician moves the radiation detection unit 20 downward and mounts the pressing panel 3 (pressing panel support unit 31) on the radiation detection unit 20. The imaging technician then switches the radiation tube to be used from the radiation tube l lb to the radiation tube 11a. As described above, in the second embodiment, when switching the mode between the mammogram imaging mode and the CBCT imaging mode, an operation of moving the radiation tube 11 and radiation detector 21 to the second side 112, which is performed in the first embodiment, is unnecessary.

As shown in FIG. 6C, preferably, the radiation detector 21a includes a large detection area 210c or two detection areas 210a and 210b, and forms narrow gaps on both the first side 111 and the second side 112. Note that a grid 22 has a grid structure suitable for mammogram imaging and CBCT imaging in each of the detection areas 210a and 210b. Note that in place of the radiation detector 21a of a large area, two radiation detectors 21 as used in the first embodiment may be arranged. However, in a case in which the two radiation detectors 21 are arranged, the imaging technician needs to switch the radiation detector to be used when switching between mammogram imaging and CBCT imaging.

According to the second embodiment as described above, the components used to move the radiation tube and the radiation detector in the rotation axis direction of the rotation frame 6 are unnecessary. Note that two radiation tubes may be arranged, as shown in FIGS. 6A to 6C, and the radiation detector 21 may be moved by the detector moving unit 23 as shown in FIGS. 5A and 5B. Alternatively, the radiation detector 21a of a large area or two radiation detectors 21 may be arranged, and the radiation tube 11 may be moved by the radiation source moving unit 12.

Note that in the above embodiments, the fixed frame 5 and the rotation frame 6 that form the rotation unit of the radiation imaging unit 2 have ring shapes. However, the present invention is not limited to this. For example, the rotation frame 6 may be a semicircular frame. For example, the outer shape of the fixed frame 5 may be not circular but rectangular. In the above embodiments, the fixed frame 5 surrounds the whole rotation frame 6. However, the present invention is not limited to this. For example, the fixed frame 5 may be formed into an arc shape and support part of the ring-shaped rotation frame 6. In the above embodiments, an example in which the radiation imaging unit 2 is mounted in the lateral direction with respect to the column 4 has been described. However, the present invention is not limited to this. The radiation imaging unit 2 need only be supported to be movable in the vertical direction with respect to the column 4. For example, the radiation imaging unit 2 may be fixed to the upper portion of the column 4.

### Third Embodiment

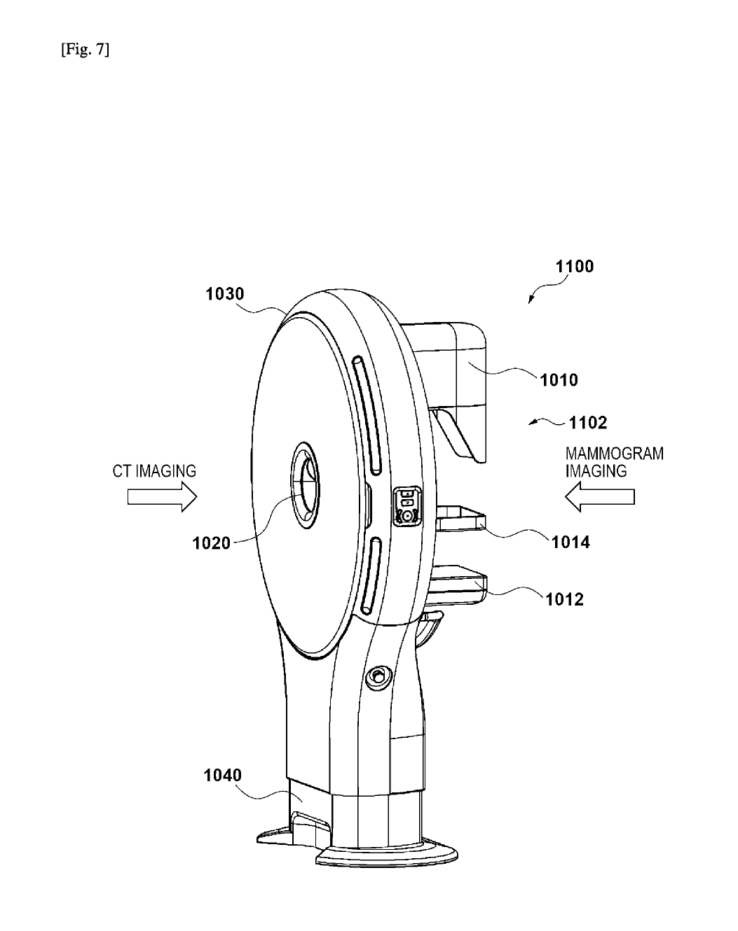

FIG. 7 is a view showing the outer appearance of a breast imaging apparatus 1100. The breast imaging apparatus 1100 can perform mammogram imaging and CT imaging. When performing mammogram imaging and CT imaging, an object is in a standing position. The standing position means a state in which the object stands on the floor with his/her feet being placed on the floor surface. That is, the breast imaging apparatus 1100 is a breast imaging apparatus for standing position.

The breast imaging apparatus 1100 includes a radiation generation unit 1010 that generates radiation, and a radiation detection unit 1012 that detects radiation irradiation from the radiation generation unit 1010, and can rotate the radiation generation unit 1010 and the radiation detection unit 1012 in a state in which they face each other. An imaging unit 1102 is mainly formed from the radiation generation unit 1010 and the radiation detection unit 1012.

Imaging is performed in a state in which a body part (breast) of an object to be imaged is sandwiched by a pressing panel 1014 from the first side of the breast imaging apparatus 1100. Here, the body part (breast) to be imaged is sandwiched between the pressing panel 1014 and the radiation detection unit 1012. Note that a grid (not shown) may be provided on the upper surface of the radiation detection unit 1012, and imaging may be performed in a state in which the body part (breast) of the object to be imaged is sandwiched between the pressing panel 1014 and the grid. That is, the breast imaging apparatus 1100 includes a first imaging unit for a mammogram imaging mode. In addition, imaging is performed while rotating the radiation generation unit 1010 and the radiation detection unit 1012 in a state in which the body part (breast) of the object to be imaged is inserted between the radiation generation unit 1010 and the radiation detection unit 1012 from the second side opposite to the first side of the breast imaging apparatus 1100. That is, the breast imaging apparatus 1100 includes a second imaging unit for a CT imaging mode.

The breast imaging apparatus 1100 also includes a gantry 1030 that rotatably supports the radiation generation unit 1010 and the radiation detection unit 1012, and a supporting leg 1040 that supports the gantry 1030 with respect to the floor surface. That is, the gantry 1030 rotatably supports the imaging unit 1102.

When performing mammogram imaging, imaging is performed in a state in which the body part (breast) of the object to be imaged is sandwiched between the pressing panel 1014 and the radiation detection unit 1012 from the first side (right side in FIG. 7) of the breast imaging apparatus 1100. The pressing panel 1014 is made of a transparent material that passes radiation. More specifically, the breast of the object can be sandwiched between the pressing panel 1014 and the radiation detection unit 1012 by moving the pressing panel 1014 in the vertical direction. In the state in which the breast of the object is sandwiched between the pressing panel 1014 and the radiation detection unit 1012, the radiation generation unit 1010 generates radiation. The radiation detection unit 1012 detects the radiation transmitted through the breast of the object, thereby capturing the breast of the object. The breast imaging apparatus 1100 can generate a mammogram based on the captured radiation data.

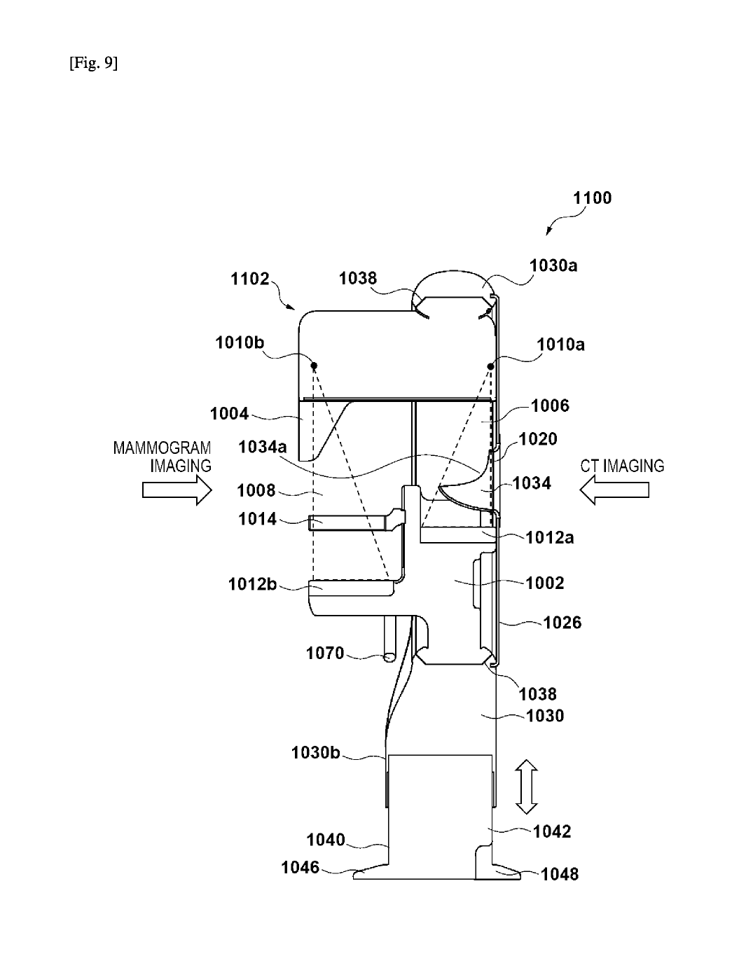

When performing CT imaging, the body part (breast) of the object to be imaged is inserted between the radiation generation unit 1010 and the radiation detection unit 1012 from the second side (left side in FIG. 7) opposite to the first side of the breast imaging apparatus 1100. In this state, imaging is performed while rotating the radiation generation unit 1010 and the radiation detection unit 1012 by a rotation frame 1038 (to be described later with reference to FIG. 9). More specifically, an opening 1020 used to insert the breast of the object is formed in the gantry 1030 of the breast imaging apparatus 1100. In the state in which the breast of the object is inserted into the opening 1020, imaging is performed while rotating the radiation generation unit 1010 and the radiation detection unit 1012 by the rotation frame 1038. During the time in which the radiation generation unit 1010 and the radiation detection unit 1012 are rotated by the rotation frame 1038, the radiation generation unit 1010 generates radiation. The radiation detection unit 1012 detects the radiation transmitted through the breast of the object, thereby capturing the breast of the object. The breast imaging apparatus 1100 can generate a CT image by reconstructing the captured radiation data.

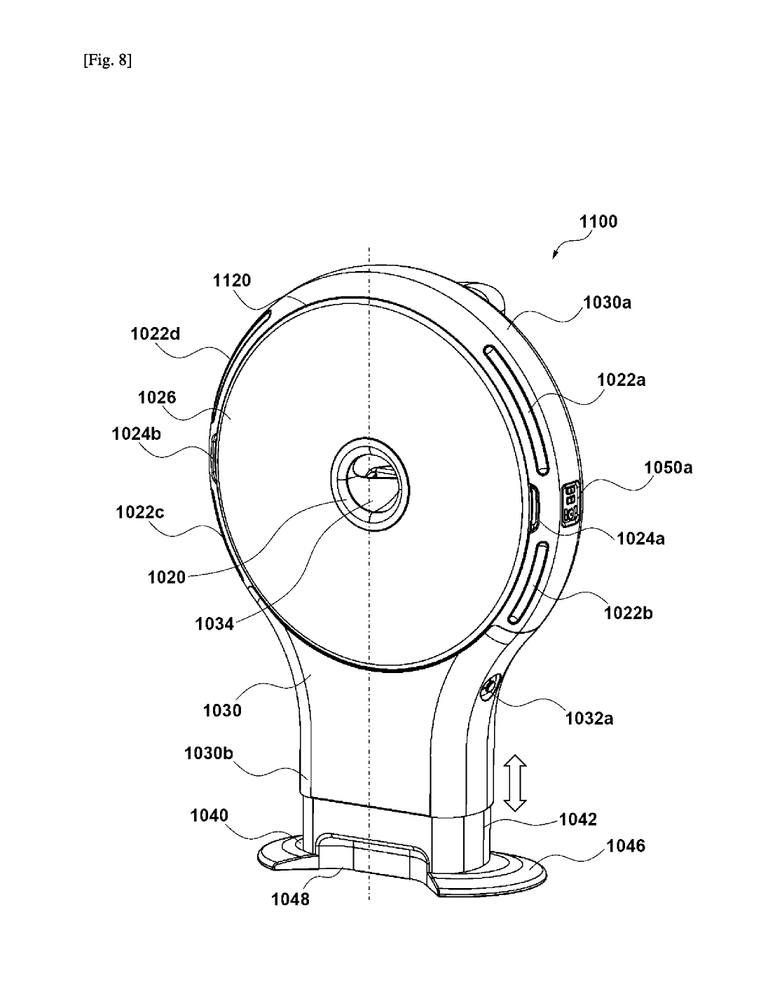

The first side of the breast imaging apparatus 1100 is the mammogram imaging side, and the second side of the breast imaging apparatus 1100 is the CT imaging side. A line that horizontally connects the first side (mammogram imaging side) and the second side (CT imaging side) is almost parallel to the rotation axis of the rotation frame 1038. In addition, the line that horizontally connects the first side (mammogram imaging side) and the second side (CT imaging side) is perpendicular to the plane of the almost flat gantry 1030 or the plane of a front cover 1026 (to be described later with reference to FIG. 8). The first side (mammogram imaging side) and the second side (CT imaging side) of the breast imaging apparatus 1100 are regions divided by the almost flat gantry 1030, the front cover 1026, and the imaging unit 1102 of the breast imaging apparatus 1100.

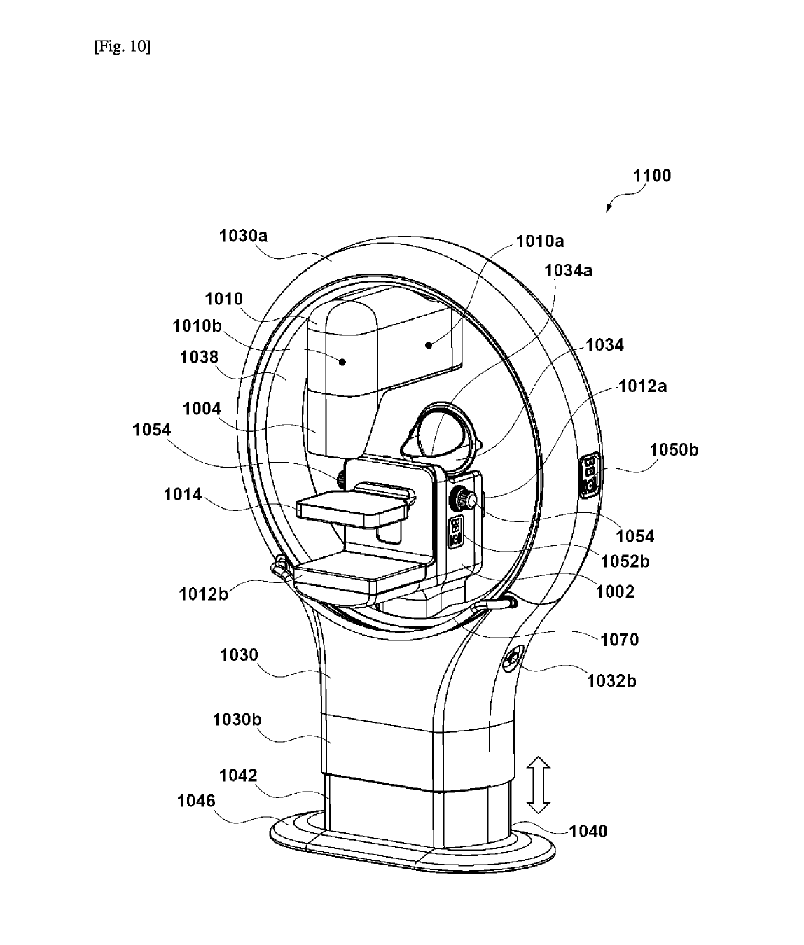

The breast imaging apparatus 1100 will be described here in detail with reference to FIGS. 8 to 10. FIG. 8 is a view showing the outer appearance of the breast imaging apparatus 1100 viewed from the CT imaging side. FIG. 9 is a sectional view of the breast imaging apparatus 1100. The sectional view of the breast imaging apparatus 1100 is a sectional view taken along the center line (alternate long and short dashed line) of the breast imaging apparatus 1100 in FIG. 8 which extends in the vertical direction. FIG. 10 is a view showing the outer appearance of the breast imaging apparatus 1100 viewed from the mammogram imaging side.

As shown in FIG. 8, the front cover 1026 that protects the object from the radiation generation unit 1010 and the radiation detection unit 1012 which rotate at the time of CT imaging is placed on the gantry 1030 on the CT imaging side. The front cover 1026 includes the opening 1020 used to insert the breast of the object undergoing CT imaging. Additionally, a plurality of grip portions 1022a, 1022b, 1022c, and 1022d to be gripped by the object undergoing CT imaging are placed on the gantry 1030 on the CT imaging side. The plurality of grip portions 1022a, 1022b, 1022c, and 1022d are formed into a concave shape. Furthermore, a notch 1048 used to insert the feet of the object undergoing CT imaging is placed on the supporting leg 1040 on the CT imaging side.

As shown in FIG. 10, the pressing panel 1014 that presses the breast of the object undergoing mammogram imaging is placed on the gantry 1030 on the mammogram imaging side. A protection plate 1004 that protects the object from unwanted exposure is placed on the gantry 1030 on the mammogram imaging side. A grip portion 1070 to be gripped by the object undergoing mammogram imaging is placed on the gantry 1030 on the mammogram imaging side. The grip portion 1070 is formed into a convex shape. The notch 1048 used to insert the feet of the object undergoing mammogram imaging is not placed on the supporting leg 1040 on the mammogram imaging side.

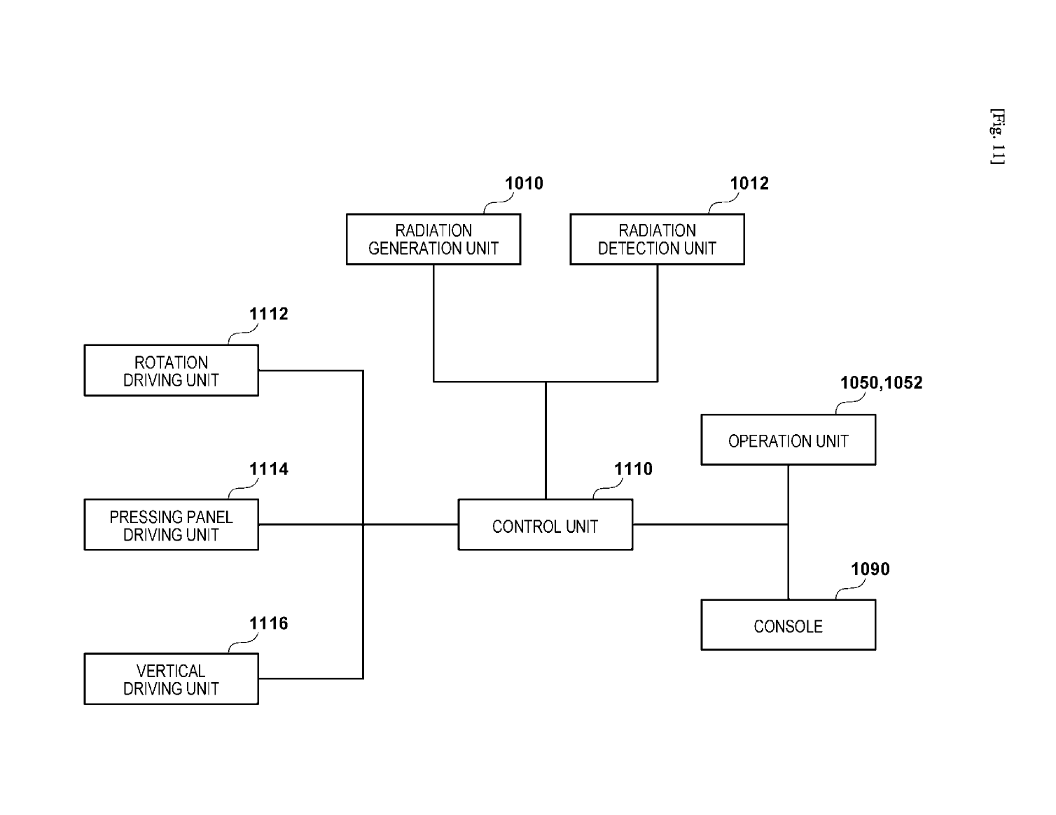

FIG. 11 is a block diagram of the breast imaging apparatus 1100. The breast imaging apparatus 1100 includes a rotation driving unit 1112 that rotates the radiation generation unit 1010 and the radiation detection unit 1012 in a state in which they face each other. The breast imaging apparatus 1100 also includes a pressing panel driving unit 1114 that vertically moves the pressing panel 1014, and a vertical driving unit 1116 that vertically moves the gantry 1030 with respect to the supporting leg 1040.

The breast imaging apparatus 1100 includes a control unit 1110 that controls the radiation generation unit 1010, the radiation detection unit 1012, the rotation driving unit 1112, the pressing panel driving unit 1114, and the vertical driving unit 1116. The breast imaging apparatus 1100 includes operation units 1050 and 1052 that transmit an instruction to the control unit 1110, and a console 1090. The operation unit 1050 that operates the breast imaging apparatus 1100 is placed on the gantry 1030. The operation unit 1052 having the same functions as the operation unit 1050 is placed on a support 1002 that supports the radiation detection unit 1012. The console 1090 is placed outside the imaging room. A display unit that displays one of the information of the object, the height information of a radiation detection unit 1012b, the dose information of a radiation generation unit 1010b, and the pressing information (N) of the pressing panel 1014 may be placed on the support 1002.

The radiation generation unit 1010 includes an electron emission source that mainly generates electrons, and a target, although not illustrated. The electrons generated by the electron emission source are emitted to the target side by the potential difference between a cathode and an anode. The target is a member that generates radiation upon electron collision. The radiation emitted by the target is shaped into a cone beam to irradiate outward. The control unit 1110 can control the imaging condition of the radiation generation unit 1010.

The radiation detection unit 1012 detects radiation transmitted through the object by a photoelectric conversion element and outputs an electric signal. For example, the radiation detection unit 1012 is formed from a conversion panel that detects the radiation transmitted through the object, a storage unit, an I/F (interface) configured to output the information converted from the radiation to the electric signal, and the like. The electric signal is output to the control unit 1110 by the I/F (interface).

### Gantry

As shown in FIGS. 8 to 10, the gantry 1030 includes the ring-shaped rotation frame 1038 configured to rotate the radiation generation unit 1010 and the radiation detection unit 1012 in a state in which they face each other, and a ring-shaped fixed frame 1030a that rotatably supports the rotation frame 1038. Note that the fixed frame 1030a may have an arc shape and rotatably support part of the rotation frame 1038. The gantry 1030 also includes an elliptic tubular portion 1030b having an elliptic tubular shape and connected to the fixed frame 1030a. The rotation frame 1038 and the fixed frame 1030a may also be called a rotation unit that rotates the radiation generation unit 1010 and the radiation detection unit 1012. The fixed frame 1030a and the elliptic tubular portion 1030b are integrally formed. The fixed frame 1030a is located above the elliptic tubular portion 1030b. The elliptic tubular portion 1030b is connected to the supporting leg 1040 that supports the gantry 1030 with respect to the floor surface.

The gantry 1030 is vertically arranged so as to do imaging of the object in a standing position. The rotation axis of the rotation unit (the rotation frame 1038 in the gantry 1030) that rotates the radiation generation unit 1010 and the radiation detection unit 1012 extends in the horizontal direction. The elliptic tubular portion 1030b covers the outer surface of an elliptic tubular portion 1042 of the supporting leg 1040. That is, the elliptic tubular portion 1042 of the supporting leg 1040 is incorporated in the elliptic tubular portion 1030b of the gantry 1030. The elliptic tubular portion 1042 of the supporting leg 1040 and the elliptic tubular portion 1030b of the gantry 1030 have a nested structure. The breast imaging apparatus 1100 includes the vertical driving unit 1116 that vertical moves the elliptic tubular portion 1030b with respect to the supporting leg 1040. That is, the breast imaging apparatus 1100 includes the vertical driving unit 1116 that vertical moves the gantry 1030.

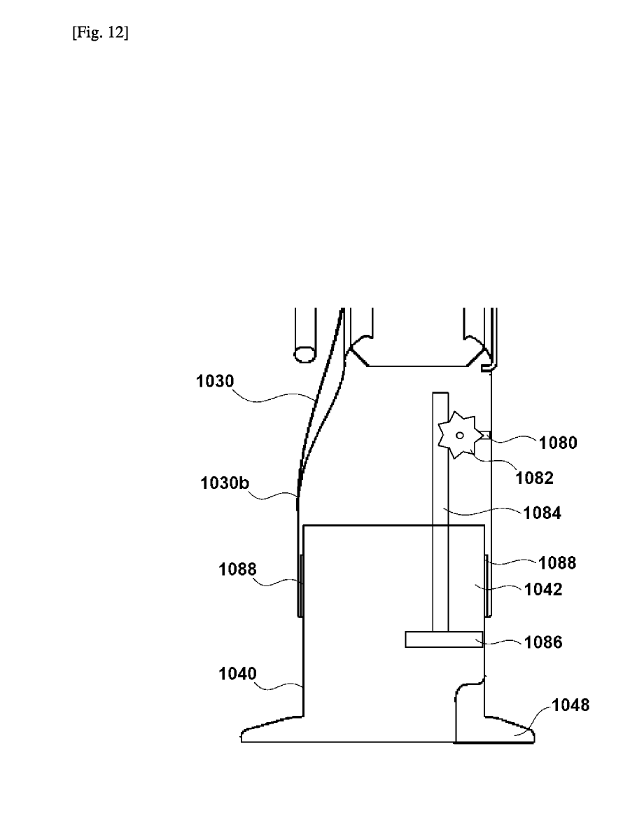

FIG. 12 is a view showing the arrangement of the vertical driving unit 1116 that vertical moves the gantry 1030. The interior of the gantry 1030 and the interior of the supporting leg 1040 are hollow. A circular gear 1082 and a support 1080 that rotatably supports the circular gear 1082 are placed inside the gantry 1030. The rotation axis of the circular gear 1082 is parallel to a horizontal plane. The support unit 1080 supports the circular gear 1082 such that the rotation axis of the circular gear 1082 becomes parallel to the horizontal plane. The gantry 1030 incorporates a driving unit (motor) that rotates the circular gear 1082, although not illustrated.

A plate-shaped rod member 1084 and a support unit 1086 that supports the rod member 1084 are placed in the supporting leg 1040. The plate-shaped rod member 1084 is placed along the longitudinal direction (vertical direction) of the supporting leg 1040. The longitudinal direction of the supporting leg 1040 is a direction perpendicular to the horizontal plane. The plate-shaped rod member 1084 is placed across the interior of the supporting leg 1040 and the interior of the gantry 1030. Note that the breast imaging apparatus 1100 may include a cushioning material 1088 that fills the gap between the elliptic tubular portion 1030b of the gantry 1030 and the elliptic tubular portion 1042 of the supporting leg 1040 to suppress play between the elliptic tubular portion 1030b of the gantry 1030 and the elliptic tubular portion 1042 of the supporting leg 1040.

The circular gear 1082 and the rod member 1084 engage with each other. When a turning force is applied to the circular gear 1082 by the driving unit (motor), the rod member 1084 can be moved vertically. More specifically, when the driving unit (motor) rotates the circular gear 1082 counterclockwise, the rod member 1084 can be moved downward with respect to the gantry 1030. When the circular gear 1082 is rotated counterclockwise, the distance between the support unit 1080 that supports the circular gear 1082 and the support unit 1086 that supports the rod member 1084 increases. When the circular gear 1082 in the vertical driving unit 1116 is thus rotated counterclockwise, the gantry 1030 can be moved upward.

In addition, when the driving unit (motor) rotates the circular gear 1082 clockwise, the rod member 1084 can be moved upward with respect to the gantry 1030. When the circular gear 1082 is rotated clockwise, the distance between the support unit 1080 that supports the circular gear 1082 and the support unit 1086 that supports the rod member 1084 decreases. When the circular gear 1082 in the vertical driving unit 1116 is thus rotated clockwise, the gantry 1030 can be moved downward.

As described above, the breast imaging apparatus 1100 can vertically move the gantry 1030 by the vertical driving unit 1116. By vertically moving the gantry 1030, the height of the opening 1020 can be adjusted according to the height of the breast of the object. Additionally, by vertically moving the gantry 1030, the height at which the breast of the object is sandwiched between the pressing panel 1014 and the radiation detection unit 1012 can be adjusted according to the height of the breast of the object. Note that as for the vertical driving unit 1116, an example of a rack and pinion system has been described above. However, any other form such as the combination of a cam follower and a guide rail may be employed.

### Radiation Generation Unit and Radiation Detection Unit

The breast imaging apparatus 1100 includes the radiation generation unit 1010 that generates radiation, and the radiation detection unit 1012 that detects radiation irradiation from the radiation generation unit 1010, and can rotate the radiation generation unit 1010 and the radiation detection unit 1012 in a state in which they face each other.

The radiation generation unit 1010 and the radiation detection unit 1012 are placed on the rotation frame 1038 that rotates with respect to the fixed frame 1030a of the gantry 1030. Here, as shown in FIG. 9, the breast imaging apparatus 1100 includes a radiation generation unit 1010a and a radiation detection unit 1012a for CT imaging, and the radiation generation unit 1010b and the radiation detection unit 1012b for mammogram imaging. That is, the breast imaging apparatus 1100 includes two sets of radiation generation units and radiation detection units for CT imaging and mammogram imaging.

The gantry 1030 includes the ring-shaped rotation frame 1038 configured to rotate the radiation generation unit 1010a and the radiation detection unit 1012a for CT imaging in a state in which they face each other and rotate the radiation generation unit 1010b and the radiation detection unit 1012b for mammogram imaging in a state in which they face each other. More specifically, the radiation generation unit 1010a and the radiation detection unit 1012a are placed on the rotation frame 1038 for CT imaging. The radiation detection unit 1012a is placed on the rotation frame 1038 via the support 1002 that supports the radiation detection unit 1012a. In addition, the radiation generation unit 1010b and the radiation detection unit 1012b are placed on the rotation frame 1038 for mammogram imaging. The radiation detection unit 1012b is placed on the rotation frame 1038 via the support 1002.

The rotation frame 1038 is connected to the fixed frame 1030a of the gantry 1030 via a bearing having a bearing structure. The fixed frame 1030a is a stationary frame in an immovable state. The rotation frame 1038 can be rotated by the rotation driving unit 1112. The rotation driving unit 1112 is placed inside the gantry 1030 such that the rotation axis of the rotation frame 1038 extends in the horizontal direction.

The pressing panel 1014 is placed on the support 1002 so as to be movable in the vertical direction. A rotation knob 1054 used to vertically move the pressing panel 1014 is placed on the support 1002. When the pressing panel 1014 is moved downward by rotating the rotation knob 1054, the breast of the object can be sandwiched between the pressing panel 1014 and the radiation detection unit 1012b. As described above, the support 1002 is placed on the rotation frame 1038. The support 1002 supports the radiation detection unit 1012a, the radiation detection unit 1012b, and the pressing panel 1014. When the rotation frame 1038 is rotated together with the support 1002 by the rotation driving unit 1112, the radiation detection units 1012a and 1012b can be rotated. In addition, when the rotation frame 1038 is rotated by the rotation driving unit 1112, the radiation generation units 1010a and 1010b can be rotated.

As shown in FIG. 9, the radiation generation units 1010a and 1010b are placed at almost the same height. The radiation detection unit 1012a is placed at a position higher than the radiation detection unit 1012b. In other words, the radiation generation units 1010a and 1010b are placed such that they are located at the same position (same distance) with respect to the rotation axis of the rotation unit (rotation frame 1038). The radiation detection units 1012a and 1012b are placed such that the radiation detection unit 1012b is located outside the radiation detection unit 1012 with respect to the rotation axis of the rotation unit (rotation frame 1038).

The distance between the radiation generation unit 1010a and the radiation detection unit 1012a used to perform CT imaging is shorter than the distance between the radiation generation unit 1010b and the radiation detection unit 1012b used to perform mammogram imaging. When performing mammogram imaging, the breast of the object is sandwiched between the pressing panel 1014 and the radiation detection unit 1012b. When pressed, the breast of the object is flattened. It is therefore necessary to increase the area of radiation irradiation and ensure an FOV (Field Of View: irradiation field). For this purpose, the radiation detection unit 1012b used to do mammogram imaging is placed at a position lower than the radiation detection unit 1012a used to do CT imaging.

An irradiation field 1008 is an irradiation field by the radiation generation unit 1010b for mammogram imaging. The radiation generation unit 1010b and the radiation detection unit 1012b are placed such that the irradiation field 1008 from the radiation generation unit 1010b includes the pressing panel 1014. The irradiation field 1008 has a quadrangular pyramid shape (cone beam shape) spreading with the apex at the focus of the radiation generation unit 1010b. As shown in FIG. 9, one end (left side) of the irradiation field 1008 is vertical, and the other end (right side) of the irradiation field 1008 is oblique. To capture the breast periphery (armpit) of the object, the irradiation field 1008 of the radiation generation unit 1010b is set such that the end (the irradiation field end or irradiation field end face) of the irradiation field 1008 on the side (left side) of the object undergoing mammogram imaging becomes vertical.

On the other hand, the radiation generation unit 1010a and the radiation detection unit 1012a are placed such that the size of the rotation frame 1038 and the size of the entire breast imaging apparatus 1100 (gantry 1030) become compact when performing CT imaging. More specifically, the radiation generation unit 1010a and the radiation detection unit 1012a are placed so as to make the distance between them as short as possible. The radiation detection unit 1012a is placed immediately under a breast holding portion 1034. The radiation detection unit 1012a is placed at a position not to contact the breast holding portion 1034 even when rotated by the rotation frame 1038.

An irradiation field 1006 is an irradiation field by the radiation generation unit 1010a for CT imaging. The breast of the object undergoing CT imaging is held on the breast holding portion 1034 and is not pressed. The radiation generation unit 1010a and the radiation detection unit 1012a are placed such that the irradiation field 1006 from the radiation generation unit 1010a includes the distal end of the breast holding portion 1034. The irradiation field 1006 has a quadrangular pyramid shape (cone beam shape) spreading with the apex at the focus of the radiation generation unit 1010a. As shown in FIG. 9, one end (right side) of the irradiation field 1006 is vertical, and the other end (left side) of the irradiation field 1006 is oblique. To capture the breast periphery (armpit) of the object, the irradiation field 1006 is set such that the end (the irradiation field end or irradiation field end face) of the irradiation field 1006 on the side (right side) of the object undergoing CT imaging becomes vertical.

As described above, the end (the irradiation field end or irradiation field end face) of the irradiation field 1008 on the side (left side) of the object undergoing mammogram imaging is set to be vertical, and the end (the irradiation field end or irradiation field end face) of the irradiation field 1006 on the side (right side) of the object undergoing CT imaging is set to be vertical. A breast cancer may metastasize to a breast periphery (armpit). To enable imaging of the breast periphery (armpit) of the object, the irradiation field 1006 of the radiation generation unit 1010a for CT imaging and the irradiation field 1008 of the radiation generation unit 1010b for mammogram imaging are set. Note that to ensure the FOV, the radiation generation unit 1010a for CT imaging may be located at a position higher than the radiation generation unit 1010b for mammogram imaging. When performing CT imaging, the radiation generation unit 1010a and the radiation detection unit 1012a are rotated while causing the radiation generation unit 1010a to generate radiation from the focus.

As described above, the breast imaging apparatus 1100 includes the first radiation generation unit 1010a that generates radiation, and the second radiation generation unit 1010b that generates radiation. The breast imaging apparatus 1100 also includes the first radiation detection unit 1012a that detects radiation irradiation from the first radiation generation unit 1010a, and the second radiation detection unit 1012b that detects radiation irradiation from the second radiation generation unit 1010b. In a state in which the body part of the object to be imaged is sandwiched between the pressing panel 1014 and the first radiation detection unit 1012a from the first side of the breast imaging apparatus 1100, imaging is performed using the first radiation generation unit 1010a and the first radiation detection unit 1012a. Additionally, in a state in which the body part of the object to be imaged is inserted between the second radiation generation unit 1010b and the second radiation detection unit 1012b from the second side opposite to the first side of the breast imaging apparatus 1100, imaging is performed while rotating the second radiation generation unit 1010b and the second radiation detection unit 1012b.

As described above, the breast imaging apparatus 1100 includes the two sets of radiation generation units and radiation detection units for CT imaging and mammogram imaging. It is therefore possible to ensure an FOV appropriate for each the breast of the object undergoing CT imaging and the breast of the object undergoing mammogram imaging.

### Rotation Frame

The breast imaging apparatus 1100 includes the rotation driving unit 1112 that rotates the radiation generation unit 1010 and the radiation detection unit 1012 via the rotation frame 1038. The radiation generation unit 1010 incorporates the second radiation generation unit 1010b for mammogram imaging and the radiation generation unit 1010a for CT imaging.

FIG. 10 shows a form to perform mammogram imaging of CC (Caranio Caudal view) by the breast imaging apparatus 1100. The position of the rotation frame 1038 is set such that the radiation generation unit 1010b, the pressing panel 1014, and the radiation detection unit 1012b are arranged in the vertical direction. By rotating the rotation knob 1054, the pressing panel 1014 can be moved to adjust the distance between the pressing panel 1014 and the radiation detection unit 1012b. The breast of the object can be pressed by moving the pressing panel 1014. In mammogram imaging of CC shown in FIG. 10, the breast arranged between the pressing panel 1014 and the radiation detection unit 1012b is pressed between the pressing panel 1014 and the radiation detection unit 1012b and undergoes radiation imaging.

The rotation driving unit 1112 is placed inside the fixed frame 1030a. The rotation frame 1038 is rotatably connected to the rotation driving unit 1112 via a connecting member (for example, a belt). A bearing is set in the gap between the fixed frame 1030a and the rotation frame 1038. When the rotation driving unit 1112 is driven, the rotation frame 1038 rotates with respect to the fixed frame 1030a.

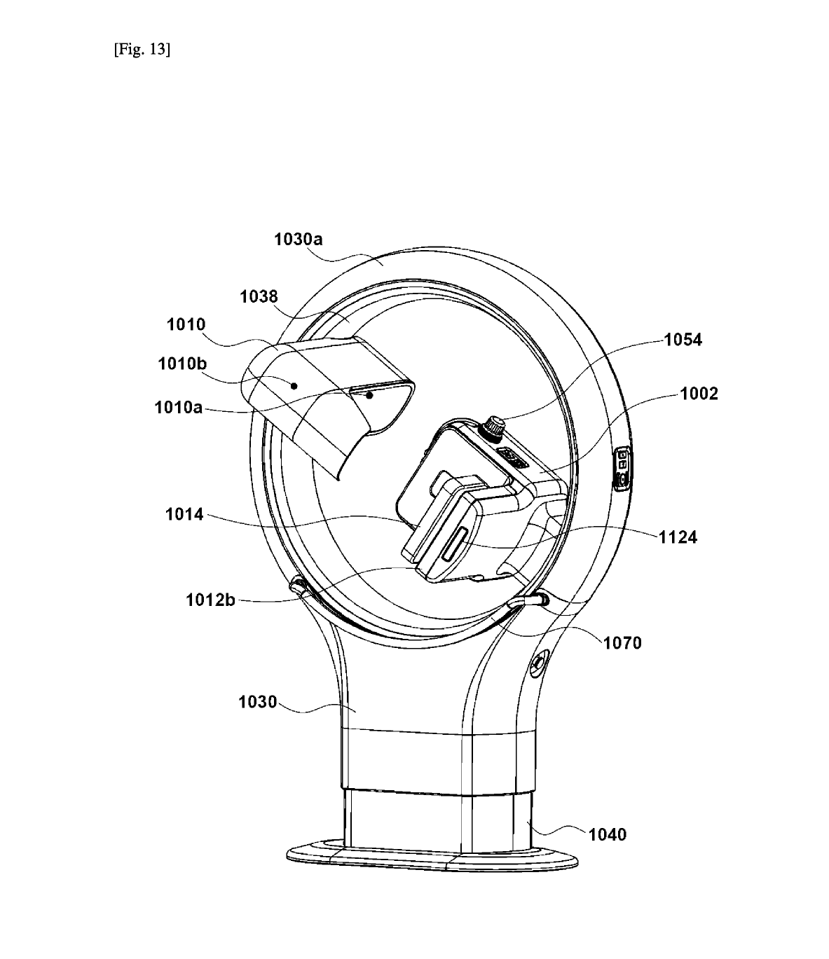

FIG. 13 is a view showing a rotation form in which the radiation generation unit 1010b and the radiation detection unit 1012b are rotated by the rotation driving unit 1112 of the breast imaging apparatus 1100. When performing mammogram imaging of MLO (MedioLateral Oblique view) by the breast imaging apparatus 1100, the rotation frame 1038 is rotated by a predetermined angle (for example, about 65°) from the state shown in FIG. 10 and stopped, as shown in FIG. 13. Note that the stop state of the rotation frame 1038 may be maintained by servo control or a brake. The breast arranged between the pressing panel 1014 and the radiation detection unit 1012b in the mammogram imaging of MLO shown in FIG. 13 is pressed and arranged between the pressing panel 1014 and the radiation detection unit 1012b and undergoes radiation imaging.

When performing CT imaging, the rotation frame 1038 is rotated with respect to the fixed frame 1030a by driving the rotation driving unit 1112. More specifically, the rotation frame 1038 is rotated by at least 180°. During the time when the rotation frame 1038 is being rotated, the radiation generation unit 1010a generates radiation, and the radiation detection unit 1012a detects the radiation. The radiation detection unit 1012 detects the radiation transmitted through the breast of the object, thereby performing CT imaging of the breast of the object. The breast imaging apparatus 1100 can generate a CT image by reconstructing the captured radiation data.

### Front Cover

The front cover 1026 is removably placed on the gantry 1030 of the breast imaging apparatus 1100. In other words, the front cover 1026 is removably placed on the ring-shaped fixed frame 1030a. More specifically, the projecting portion of the front cover 1026 is fitted in the frame body (groove portion) of the fixed frame 1030a, thereby mounting the front cover 1026 on the fixed frame 1030a.

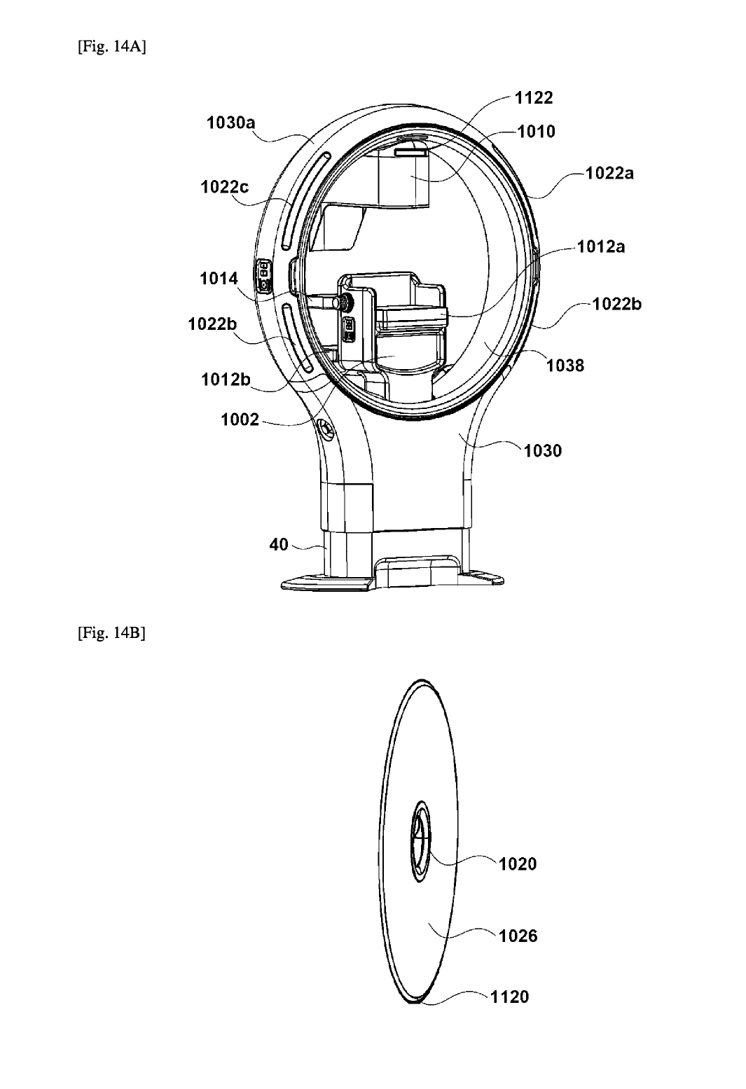

FIG. 8 shows a state in which the front cover 1026 is mounted on the gantry 1030 of the breast imaging apparatus 1100. FIGS. 14A and 14B show a state in which the front cover 1026 is removed from the gantry 1030 of the breast imaging apparatus 1100. When the front cover 1026 is removed from the gantry 1030, as shown in FIGS. 14A and 14B, the operator can access the breast of the object undergoing mammogram imaging from the CT imaging side via the hollow portion of the rotation frame 1038. In mammogram imaging, position adjustment and pressing adjustment can be done for the breast arranged between the radiation detection unit 1012b and the pressing panel 1014 of the breast imaging apparatus 1100.

Since the fixed frame 1030a of the gantry 1030 has a ring shape, the front cover 1026 is circular. The front cover 1026 need only be fixed to a member immovable with respect to the rotation of the radiation generation unit 1010 and the radiation detection unit 1012. The opening 1020 used to insert the breast of the object is placed in the front cover 1026. More specifically, as shown in FIG. 8, the circular opening 1020 used to insert the breast of the object is placed at the center of the front cover 1026.

The front cover 1026 is formed from a shield member that shields radiation. In CT imaging or mammogram imaging, scattered radiation does not pass through the front cover 1026. That is, scattered radiation is prevented from reaching the CT imaging side. The front cover 1026 is a semitransparent member. Since the front cover 1026 is semitransparent, the position of the radiation generation unit 1010 or the radiation detection unit 1012 can be confirmed from the CT imaging side even if the front cover 1026 is placed on the gantry 1030.

As shown in FIGS. 9 and 10, the breast holding portion 1034 serving as a holder to hold the breast inserted from the opening 1020 is placed in the front cover 1026. The breast holding portion 1034 is a transparent member that passes radiation. The breast holding portion 1034 is formed along the periphery of the opening 1020 of the front cover 1026. The breast holding portion 1034 is formed to project from the opening 1020 of the front cover 1026 to the side of the imaging unit 1102. The breast holding portion 1034 is hollow and is curved in conformity with the shape of the breast. Part (lower side) of the breast holding portion 1034 has a cup shape, and part (upper side) of the breast holding portion 1034 is open. The breast holding portion 1034 has an opening 1034a used by the operator to access the breast from the mammogram imaging side. The breast holding portion 1034 has a shape that holds the lower side of the breast. The breast holding portion 1034 can hold the breast while keeping its shape. The breast holding portion 1034 does not press the breast.

As shown in FIG. 10, since part (upper side) of the breast holding portion 1034 is open, the operator can access the breast of the object undergoing CT imaging from the mammogram imaging side. That is, the operator can access the breast of the object held on the breast holding portion 1034 from the mammogram imaging side. The breast holding portion 1034 is removable from the front cover 1026. Here, breast holding portions 1034 of various sizes or shapes can be prepared. That is, the operator can prepare a plurality of breast holding portions 1034. For example, the breast holding portion 1034 can be exchanged in accordance with the size of the breast of the object.

The breast holding portion 1034 and the front cover 1026 fixed to the gantry 1030 separate the space of the object from the radiation generation unit 1010 and the radiation detection unit 1012 which rotate during CT imaging. The breast of the object is held on the breast holding portion 1034 and therefore fixed during imaging.

An example in which the breast holding portion 1034 is connected along the periphery of the opening 1020 of the front cover 1026 has been described above. However, the present invention is not limited to this. For example, the breast holding portion 1034 may be connected to the fixed frame 1030a of the gantry 1030.

In addition, insertion portions 1024a and 1024b are placed in the gantry 1030. The insertion portions 1024a and 1024b are portions used to insert the operator's hands when removing the front cover 1026, and are placed along the fixed frame 1030a of the gantry 1030. The insertion portions 1024a and 1024b are placed at two, right and left portions of the fixed frame 1030a of the gantry 1030. The insertion portions 1024a and 1024b are placed so as to be symmetric with respect to the center line (alternate long and short dashed line) of the gantry 1030.

The operator inserts the hands into the insertion portions 1024a and 1024b and pulls the front cover 1026 to the near side, thereby removing the front cover 1026. When the front cover 1026 is removed at the time of mammogram imaging, the operator can easily access the breast of the object. To the contrary, the operator presses the front cover 1026 to the far side into the fixed frame 1030a of the gantry 1030, thereby mounting the front cover 1026 on the gantry 1030.

Note that the front cover 1026 may be opaque from the side of the object undergoing CT imaging and transparent from the operator side on the mammogram imaging side. The front cover 1026 formed to be opaque from the object side can prevent the object from becoming frightened by viewing the movement of the radiation generation unit 1010 or the radiation detection unit 1012 through the front cover 1026.

As shown in FIGS. 8, 14A, and 14B, a first illumination unit 1120 that irradiates the CT imaging side with light is placed on the front cover 1026. For example, the first illumination unit 1120 is placed along the circumferential direction of the front cover 1026. The first illumination unit 1120 may be placed on the gantry 1030 (the fixed frame 1030a and the elliptic tubular portion 1030b) on the CT imaging side. That is, the first illumination unit 1120 is placed for light irradiation from the breast imaging apparatus 1100 on the CT imaging side. The near side of the breast imaging apparatus 1100 shown in FIG. 8 need only be irradiated with light. Light irradiation from the first illumination unit 1120 can employ either a lighting form or a blinking form. The illumination unit is controlled by the control unit 1110, although not illustrated.

When performing CT imaging, the control unit 1110 causes the first illumination unit 1120 to irradiate the CT imaging side with light. When performing CT imaging, the first illumination unit 1120 irradiates the CT imaging side with light. Hence, the object scheduled to undergo CT imaging can recognize which side of the breast imaging apparatus is to be used for the imaging. During CT imaging, the first illumination unit 1120 turns off the radiation irradiation on the CT imaging side. “During CT imaging” means the period in which CT imaging is being performed by rotating the radiation generation unit 1010 and the radiation detection unit 1012. That is, radiation irradiation from the first illumination unit 1120 is absent during CT imaging.

A second illumination unit 1122 that irradiates the CT imaging side with light is placed on the radiation generation unit 1010. The control unit 1110 causes the second illumination unit 1122 to irradiate the CT imaging side with light. Light irradiation from the second illumination unit 1122 employs a lighting form. Since the light irradiation from the second illumination unit 1122 employs a lighting form, the position of the radiation generation unit 1010 or the radiation detection unit 1012 can be confirmed even if the radiation generation unit 1010 and the radiation detection unit 1012 rotate.

Additionally, since the front cover 1026 is semitransparent, the second illumination unit 1122 irradiates the CT imaging side with light. During CT imaging in which the radiation generation unit 1010 and the radiation detection unit 1012 are rotating, the radiation generation unit 1010 generates radiation, and the second illumination unit 1122 performs radiation irradiation. The operator can confirm the position of the rotating radiation generation unit 1010 or radiation detection unit 1012 from the CT imaging side. Hence, by confirming the position of the radiation generation unit 1010 or radiation detection unit 1012 from the CT imaging side, the operator can recognize how much the radiation generation unit 1010 should rotate to end the CT imaging. Note that a form in which the second illumination unit 1122 that irradiates the CT imaging side with light is placed on the radiation generation unit 1010 has been described. However, the second illumination unit 1122 may be placed on the radiation detection unit 1012.

When the radiation generation unit 1010 and the radiation detection unit 1012 rotate, and CT imaging ends, the control unit 1110 causes the first illumination unit 1120 to irradiate the CT imaging side with light. The first illumination unit 1120 irradiates the CT imaging side with light. Hence, the operator and the object can recognize that the CT imaging has ended.

In addition, as shown in FIG. 13, a third illumination unit 1124 that irradiates the mammogram imaging side with light is placed on the radiation generation unit 1010, the radiation detection unit 1012, and the pressing panel 1014. The third illumination unit 1124 may be placed on the gantry 1030 (the fixed frame 1030a and the elliptic tubular portion 1030b) on the mammogram imaging side. That is, the third illumination unit 1124 is placed such that radiation irradiation is done from the breast imaging apparatus on the mammogram imaging. It is only necessary to irradiate the near side of the breast imaging apparatus shown in FIG. 13 with light. Light irradiation from the third illumination unit 1124 can employ either a lighting form or a blinking form.

When performing mammogram imaging, the control unit 1110 causes the third illumination unit to irradiate the mammogram imaging side with light. When performing mammogram imaging, the third illumination unit 1124 irradiates the mammogram imaging side with light. Hence, the object scheduled to undergo mammogram imaging can recognize which side of the breast imaging apparatus is to be used for the imaging.

As described above, the front cover 1026 that protects the object from the radiation generation unit 1010 and the radiation detection unit 1012 which rotate at the time of CT imaging is removably placed on the gantry 1030 of the breast imaging apparatus 1100 according to this embodiment. The front cover 1026 includes the opening 1020 used to insert the breast of the object and the breast holding portion 1034 that holds the breast of the object inserted from the opening 1020. It is therefore possible to protect the object from the radiation generation unit 1010 and the radiation detection unit 1012 which rotate at the time of CT imaging.

### Supporting Leg (Notch)

As shown in FIGS. 8 to 10, the breast imaging apparatus 1100 includes the gantry 1030 that rotatably supports the imaging unit 1102, and the supporting leg 1040 that supports the breast imaging apparatus 1100 (gantry 1030) with respect to the floor surface. The supporting leg 1040 supports the breast imaging apparatus 1100 (gantry 1030) in the vertical direction.

The supporting leg 1040 is formed from the elliptic tubular portion 1042 formed to extend in a direction (vertical direction) perpendicular to the horizontal plane, and a base 1046 connected to the elliptic tubular portion 1042 and configured to stably support the breast imaging apparatus 1100 (gantry 1030). The base 1046 is a member that comes into contact with the floor surface. The elliptic tubular portion 1042 and the base 1046 of the supporting leg 1040 are integrated. The gantry 1030 is connected to the elliptic tubular portion 1042.

The base 1046 is in contact with the floor surface. The base 1046 is formed in conformity with the peripheral shape of the elliptic tubular portion 1042. For example, the elliptic tubular portion 1042 has an elliptic tubular form made by connecting two semicircles by lines. Since the elliptic tubular portion 1042 has the elliptic tubular form, the base 1046 is formed into an elliptic shape. The elliptic tubular portion 1042 and the base 1046 are similar to each other.

More specifically, the base 1046 is formed so as to project outward from the periphery of the bottom surface of the elliptic tubular portion 1042. In other words, the base 1046 is formed so as to evenly project outward from the periphery of the bottom surface of the elliptic tubular portion 1042. The base 1046 is formed so as to project by a predetermined width (for example, about 10 to 30 cm) outward from the periphery of the bottom surface of the elliptic tubular portion 1042. The contact area between the base 1046 and the floor surface is larger than the area of the bottom surface of the elliptic tubular portion 1042. By extending the diameter of the bottom surface of the elliptic tubular portion 1042 by the base 1046, the breast imaging apparatus 1100 can increase the contact area to the floor surface. With the base 1046, the placement balance of the breast imaging apparatus 1100 (gantry 1030) can be maintained, and the breast imaging apparatus 1100 (gantry 1030) can stably be supported.

The tips (toes) of the feet of the object are inserted into the notch 1048. The notch 1048 is opened so much as to receive the tips (toes) of both feet of the object. More specifically, the notch 1048 is formed by cutting part of the base 1046 on a predetermined side. In other words, the notch 1048 is formed by making a hole inside the base 1046 on the predetermined side. For example, the depth of the notch 1048 is about 20 cm, and the height of the notch 1048 is about 10 cm from the floor surface. Note that the notch 1048 may be formed by cutting part of the elliptic tubular portion 1042 on the predetermined side together with the base 1046. The predetermined side is the CT imaging side on which the object is arranged when performing CT imaging. The CT imaging side is the near side of the breast imaging apparatus 1100 shown in FIG. 8 or the right side of the breast imaging apparatus 1100 shown in FIG. 9.

As shown in FIG. 9, when performing CT imaging, imaging is performed by rotating the radiation generation unit 1010 and the radiation detection unit 1012 by the rotation frame 1038 in a state in which the breast of the object is inserted into the opening 1020 of the front cover 1026. When performing CT imaging, the object faces the breast imaging apparatus 1100 with the upper body adhered to the front cover 1026.

A vertical line drawn from the end face of the front cover 1026 reaches the base 1046. For this reason, if the base 1046 does not have the notch 1048, the object needs to place the feet on the base 1046. The object undergoes CT imaging in an unstable state. To prevent this, the breast imaging apparatus 1100 according to the present invention includes the notch 1048 in the base 1046 used to insert the feet of the object. The notch 1048 is formed in the base 1046 so as to expose the floor surface. Hence, the object inserts the feet into the notch 1048 and places them on the floor surface, thereby maintaining the posture in a state in which the object faces the breast imaging apparatus 1100. The object undergoes CT imaging in a stable state.

Considering the placement balance of the breast imaging apparatus 1100 (gantry 1030), the notch 1048 is formed without cutting the base 1046 on the side opposite to the predetermined side. In other words, the notch 1048 is formed in the supporting leg 1040 so as not to lose the placement balance of the breast imaging apparatus 1100 (gantry 1030). The base 1046 projects similarly on the CT imaging side and the mammogram imaging side. While ensuring the projecting portion of the base 1046, the notch 1048 used to insert the feet of the object is provided in the base 1046 on the CT imaging side.

On the other hand, as shown in FIGS. 8 to 10, the notch 1048 used to insert the feet of the object is not provided in the base 1046 on the mammogram imaging side where the object is arranged when performing mammogram imaging. In other words, the notch 1048 is not provided in the supporting leg 1040 on the mammogram imaging side. That is, the notch 1048 is provided in the supporting leg 1040 only on the CT imaging side (right side). This is because on the mammogram imaging side, constituent elements such as the radiation generation unit 1010b, the radiation detection unit 1012b, and the pressing panel 1014 project to the mammogram imaging side (left side), that is, the side (left side) of the object undergoing mammogram imaging. The constituent elements such as the radiation generation unit 1010b, the radiation detection unit 1012b, and the pressing panel 1014 project from the position of the base 1046 to the side (left side) of the object undergoing mammogram imaging. Hence, the notch 1048 need not be provided in the base 1046 on the mammogram imaging side.

As described above, the breast imaging apparatus 1100 includes the supporting leg 1040 that supports the gantry 1030 with respect to the floor surface. The supporting leg 1040 includes the notch 1048 used to insert the feet of the object. Imaging is performed by rotating the radiation generation unit 1010 and the radiation detection unit 1012 in a state in which the body part of the object to be imaged is inserted between the radiation generation unit 1010 and the radiation detection unit 1012. The notch 1048 used to insert the feet of the object is formed in the supporting leg 1040 on the CT imaging side where the object is arranged at that time.

The notch used to insert the feet of the object is not formed in the supporting leg 1040 on the mammogram imaging side where the object is arranged when imaging is performed using the radiation generation unit 1010 and the radiation detection unit 1012 in a state in which the body part of the object to be imaged is sandwiched between the pressing panel 1014 and the radiation detection unit 1012. Hence, the object can take a posture suitable for each imaging.

### Grip Portion

As shown in FIG. 8, the plurality of grip portions 1022a, 1022b, 1022c, and 1022d to be gripped by the object undergoing CT imaging are formed in the gantry 1030 on the CT imaging side. More specifically, the plurality of grip portions 1022a, 1022b, 1022c, and 1022d are formed along the circumferential direction of the outer edge of the ring-shaped fixed frame 1030a that rotatably supports the rotation frame 1038 in the gantry 1030. In addition, the plurality of grip portions 1022a, 1022b, 1022c, and 1022d are formed near the joint between the fixed frame 1030a and the front cover 1026 placed on the fixed frame 1030a.

In the fixed frame 1030a, the grip portion 1022a is placed on the upper right side, the grip portion 1022b is placed on the lower right side, the grip portion 1022c is placed on the upper left side, and the grip portion 1022d is placed on the lower left side. Although not illustrated, a grip portion may be placed atop the fixed frame 1030a. The plurality of grip portions 1022a, 1022b, 1022c, and 1022d are grip portions having a concave shape, and are formed on the ring-shaped fixed frame 1030a. The grip portions 1022 sink such that at least the tips of the fingers of the object are caught on.

As shown in FIG. 10, the grip portion 1070 to be gripped by the object undergoing mammogram imaging is placed on the gantry 1030 on the mammogram imaging side. The grip portion 1070 is formed along the circumferential direction of the outer edge of the ring-shaped fixed frame 1030a. More specifically, the grip portion 1070 is formed to project from two supporting points on the fixed frame 1030a. The supporting points are located on the lower side of the fixed frame 1030a. The distance between the two supporting points of the grip portion 1070 is longer than the width of the radiation generation unit 1010b or the radiation detection unit 1012b. In addition, the distance between the two supporting points of the grip portion 1070 is longer than the width of the support 1002.

The grip portion 1070 is a rod-shaped member and is placed into an arch shape that connects the two supporting points on the fixed frame 1030a. The grip portion 1070 is a member like a so-called handrail. The grip portion 1070 is curved. More specifically, the grip portion 1070 is curved downward. This aims at preventing the radiation generation unit 1010 and the radiation detection unit 1012 from interfering with (colliding against) the grip portion 1070 even when the radiation generation unit 1010 and the radiation detection unit 1012 are rotated. A gap is formed between the fixed frame 1030a and the arch-shaped grip portion 1070. The gap is several cm long. Since the gap exists between the fixed frame 1030a and the grip portion 1070, the object can grip the grip portion 1070 by both hands.

Since the distance between the two supporting points of the grip portion 1070 is longer than the width of the radiation generation unit 1010b or the radiation detection unit 1012b, the grip portion 1070 is not hidden by the radiation generation unit 1010 and the radiation detection unit 1012 even when the radiation generation unit 1010 and the radiation detection unit 1012 rotate. In addition, even when the radiation generation unit 1010 and the radiation detection unit 1012 rotate, the object can grip the grip portion 1070.

The plurality of grip portions 1022a, 1022b, 1022c, and 1022d on the CT imaging side and the grip portion 1070 on the mammogram imaging side have different shapes. The grip portions 1022a, 1022b, 1022c, and 1022d on the CT imaging side sink with respect to the gantry 1030, whereas the grip portion 1070 on the mammogram imaging side projects with respect to the gantry 1030.

On the CT imaging side, it is necessary to adhere the upper body of the object to the front cover 1026 and insert the breast of the object as the body part to be imaged into the opening 1020. Hence, the grip portions 1022a, 1022b, 1022c, and 1022d on the CT imaging side do not project with respect to the gantry 1030 so as not to hinder the object adhered to the front cover 1026. The grip portions 1022a, 1022b, 1022c, and 1022d on the CT imaging side are formed into a concave shape.

On the mammogram imaging side, the upper body of the object is not adhered to the front cover 1026. It is necessary to insert and sandwich the breast of the object between the pressing panel 1014 and the radiation detection unit 1012 which project from the gantry 1030. It is also necessary to support the body of the object in a state in which the breast of the object is pressed. Hence, the grip portion 1070 on the mammogram imaging side projects with respect to the gantry 1030. The grip portion 1070 on the mammogram imaging side is formed into a convex shape.

As described above, the first grip portion 1070 to be gripped by the object is provided on the gantry 1030 on the first side of the breast imaging apparatus 1100 according to the present invention. The second grip portions 1022 whose shape is different from that of the first grip portion 1070 are provided on the gantry 1030 on the second side opposite to the first side. The first grip portion 1070 is formed into a convex shape with respect to the gantry 1030, and the second grip portions 1022 are formed into a concave shape with respect to the gantry 1030. Hence, the object undergoing mammogram imaging or the object undergoing CT imaging can take a posture appropriate for the imaging by appropriately gripping the first grip portion 1070 or the second grip portions 1022.

### Operation Units

As shown in FIGS. 8 and 10, the operation unit 1050 that operates the breast imaging apparatus is placed on the gantry 1030. The operation unit 1050 is placed on the unrotatable constituent element of the breast imaging apparatus. The operation unit 1050 is placed on each of the left and right side surfaces of the fixed frame 1030a of the gantry 1030. More specifically, the operation unit 1050 is formed from operation units 1050a and 1050b. The operation units 1050a and 1050b are placed on the right and left ends of the fixed frame 1030a, respectively. As shown in FIG. 11, the operation units 1050 (operation units 1050a and 1050b) are connected to the control unit 1110.

As shown in FIG. 10, the operation unit 1052 (operation units 1052a and 1052b) that operates the breast imaging apparatus is placed on the support 1002 placed on the rotation frame 1038. That is, the operation unit 1052 is placed on the rotatable constituent element of the breast imaging apparatus. In this embodiment, the operation unit 1052 is placed on each of the left and right side surfaces of the support 1002. More specifically, the operation unit 1052 is formed from the operation units 1052a and 1052b. The operation units 1052a and 1052b are placed on the right and left ends of the support 1002, respectively. As shown in FIG. 11, the operation units 1052 (operation units 1052a and 1052b) are connected to the control unit 1110.

The operation unit 1050 placed on the gantry 1030 and the operation unit 1052 placed on the support 1002 have the same button structure. The operation units 1050 and 1052 have the same function. Each of the operation units 1050 and 1052 is formed from a button that vertically moves the pressing panel 1014 and a button that rotates the radiation generation unit 1010 or the radiation detection unit 1012. These buttons are pressed by a finger of the operator.

Note that an example in which the breast imaging apparatus 1100 according to this embodiment includes two sets of radiation generation units and radiation detection units for CT imaging and mammogram imaging has been described. However, it is also possible to implement CT imaging and mammogram imaging using one set of a radiation generation unit and a radiation detection unit. More specifically, the breast imaging apparatus 1100 is provided with a moving mechanism (not shown) that moves the radiation generation unit and the radiation detection unit in CT imaging and mammogram imaging. For example, in CT imaging, the radiation generation unit is moved to the position of the radiation generation unit 1010a shown in FIG. 9, and the radiation detection unit is moved to the position of the radiation detection unit 1012a. In mammogram imaging, the radiation generation unit is moved to the position of the radiation generation unit 1010b, and the radiation detection unit is moved to the position of the radiation detection unit 1012b shown in FIG. 9.

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2015-131836, filed Jun. 30, 2015, which is hereby incorporated by reference herein in its entirety.

1. A breast imaging apparatus including a radiation generation unit configured to generate radiation and a radiation detection unit configured to detect radiation irradiation from the radiation generation unit and capable of rotating the radiation generation unit and the radiation detection unit in a state in which the radiation generation unit and the radiation detection unit face each other, comprising:
a first imaging unit configured to perform imaging in a state in which a breast of an object is pressed by a pressing panel on a first side of the breast imaging apparatus; and
a second imaging unit configured to perform imaging while rotating the radiation generation unit and the radiation detection unit in a state in which the breast of object is inserted between the radiation generation unit and the radiation detection unit on a second side opposite to the first side of the breast imaging apparatus.

2. The apparatus according to claim 1, wherein the radiation generation unit and the radiation detection unit are supported by a column to be movable in a vertical direction.

3. The apparatus according to claim 2, wherein the column is fixed to a cart.

4. The apparatus according to claim 1, wherein a front cover is removably provided on the second side, and an opening used to insert the breast of the object is provided in the front cover.

5. The apparatus according to claim 4, wherein the front cover includes, around the opening, a support configured to support the breast of the object inserted from the opening.

6. The apparatus according to claim 1, wherein the pressing panel is removable.

7. The apparatus according to claim 1, wherein the imaging performed by inserting the breast of the object from the first side is mammogram imaging, and the imaging performed by inserting the breast of the object from the second side is CT imaging.

8. The apparatus according to claim 7, wherein for the mammogram imaging and the CT imaging, the radiation generation unit can move and arrange a radiation source of the radiation generation unit in a rotation axis direction of a rotation of the radiation generation unit and the radiation detection unit.

9. The apparatus according to claim 7, wherein for the mammogram imaging and the CT imaging, the radiation detection unit can move and arrange a detector of the radiation detection unit in a rotation axis direction of the rotation of the radiation generation unit and the radiation detection unit.

10. The apparatus according to claim 7, wherein the radiation generation unit includes a first radiation source for the mammogram imaging and a second radiation source for the CT imaging.

11. The apparatus according to claim 7, wherein the radiation detection unit includes a first detector for the mammogram imaging and a second detector for the CT imaging.

12. The apparatus according to claim 7, wherein on the first side, the radiation source of the radiation generation unit, a detector of the radiation detection unit, and the pressing panel are arranged at positions projecting to the first side with respect to a surface of revolution of a rotation of the radiation generation unit and the radiation detection unit.

13. The apparatus according to claim 7, wherein the radiation generation unit and the radiation detection unit provide different imaging geometric systems in the mammogram imaging and the CT imaging.

14. The apparatus according to claim 13, further comprising a changing unit configured to change a distance between the radiation source of the radiation generation unit and the detector of the radiation detection unit by moving the detector toward a rotation center of the rotation of the radiation generation unit and the radiation detection unit.

15. The apparatus according to claim 8, wherein the radiation generation unit can be mounted in a state in which a radiation shape from the radiation source is rotated by 180° about a radial direction from a rotation center of the rotation of the radiation generation unit and the radiation detection unit between the mammogram imaging and the CT imaging.

16. The apparatus according to claim 9, wherein the radiation detection unit can be mounted in a state in which the detector is rotated by 180° about a radial direction from a rotation center of the rotation of the radiation generation unit and the radiation detection unit between the mammogram imaging and the CT imaging.

17. The apparatus according to claim 1, wherein a gantry configured to rotate the radiation generation unit and the radiation detection unit in the state in which the radiation generation unit and the radiation detection unit face each other has a space used by an operator to access, from the first side, the breast inserted from the second side where the imaging is performed by the second imaging unit.

18. The apparatus according to claim 1, wherein a support form for supporting the breast to be captured by the second imaging unit is different from a support form for supporting the breast by the first imaging unit.

19. The apparatus according to claim 2, wherein a lower portion of a breast support is curved, and an upper portion of the breast support is open.

20. A breast imaging apparatus including a radiation generation unit configured to generate radiation and a radiation detection unit configured to detect radiation irradiation from the radiation generation unit and capable of rotating the radiation generation unit and the radiation detection unit in a state in which the radiation generation unit and the radiation detection unit face each other, comprising:
a first imaging unit configured to perform mammogram imaging while pressing a breast on a first side of the breast imaging apparatus; and
a second imaging unit configured to perform CT imaging on a second side opposite to the first side of the breast imaging apparatus.

21. A breast imaging apparatus including a radiation generation unit configured to generate radiation and a radiation detection unit configured to detect radiation irradiation from the radiation generation unit and capable of rotating the radiation generation unit and the radiation detection unit in a state in which the radiation generation unit and the radiation detection unit face each other, comprising:
a first imaging unit configured to perform mammogram imaging using a pressing panel on a first side of the breast imaging apparatus; and
a second imaging unit configured to perform CT imaging while rotating the radiation generation unit and the radiation detection unit on a second side opposite to the first side of the breast imaging apparatus.
